SEQUENCE LISTING (10 sequences)

SEQ ID NO: 1 - DNA, 23 bp, synthetic construct
  source 1..23: mol_type = other DNA
tcatccgctttcgacactcgcgg

SEQ ID NO: 2 - DNA, 22 bp, synthetic construct
  source 1..22: mol_type = other DNA
cacgatgagtattgagtcttgg

SEQ ID NO: 3 - DNA, 23 bp, synthetic construct
  source 1..23: mol_type = other DNA
tgcttcgtacagctgccagtcgg

SEQ ID NO: 4 - DNA, 23 bp, synthetic construct
  source 1..23: mol_type = other DNA
cttccaccggcggtaatctttgg

SEQ ID NO: 5 - DNA, 23 bp, synthetic construct
  source 1..23: mol_type = other DNA
ttatcacattgtatgacggtcgg

SEQ ID NO: 6 - DNA, 23 bp, synthetic construct
  source 1..23: mol_type = other DNA
gtgctggacgcattcctattggg

SEQ ID NO: 7 - DNA, 23 bp, synthetic construct
  source 1..23: mol_type = other DNA
cgacggctcgttcatcgctgggg

SEQ ID NO: 8 - DNA, 23 bp, synthetic construct
  source 1..23: mol_type = other DNA
gtcgacggctcgttcatcgctgg

SEQ ID NO: 9 - DNA, 23 bp, synthetic construct
  source 1..23: mol_type = other DNA
cttgaacagctctatcagatcgg

SEQ ID NO: 10 - DNA, 23 bp, synthetic construct
  source 1..23: mol_type = other DNA
atcgagcgcgtcgcctggtacgg

(19) United States
(12) Patent Application Publication
(10) Pub. No.: US 2025/0324957 A1
(43) Pub. Date: Oct. 23, 2025

(54) CONFINABLE POPULATION SUPPRESSION SYSTEM

(71) Applicant: The Regents of the University of California, Oakland, CA (US)

(72) Inventors: Omar Akbari, San Diego, CA (US); Andie Smidler, San Diego, CA (US); James Pai, Chino Hills, CA (US); Reema Apte, San Diego, CA (US)

(21) Appl. No.: 18/868,107

(22) PCT Filed: May 25, 2023
(86) PCT No.: PCT/US2023/067494

§ 371 Date: Nov. 21, 2024

Related U.S. Application Data
(60) Provisional application No. 63/346,207, filed on May 26, 2022.

(51) Int. Cl. A01K 67/68; C12N 9/22

(52) U.S. Cl. CPC A01K 67/68; C12N 9/226; A01K 2217/052; A01K 2217/15; A01K 2227/706

(57) ABSTRACT

Provided herein are methods and transgenic systems termed Ifegenia (Inherited Female Elimination by Genetically Encoded Nucleases to Interrupt Alleles) comprising transgenic animal strains encoding Cas9 and/or gRNA that targets a female essential gene which are capable of passing down these genes as well as mutant female essential genes in wild populations in order to suppress the population of the animals, as well as methods and systems for making such animals. In some instances, the methods and systems provided herein result in both somatic and heritable germline mutations of the female essential gene resulting in daughter killing, and female essential gene mutant males reproductively viable to pass along the female essential gene mutation and related transgenes into subsequent generations. The methods and systems are adaptable to population control of insects, in particular mosquitoes such as Anopheles gambiae.

### CROSS REFERENCE TO RELATED APPLICATIONS

This application claims the priority benefit of U.S. Provisional Application No. 63/346,207, filed May 26, 2022, which application is incorporated herein by reference.

### GOVERNMENT SPONSORSHIP

This invention was made with government support under AI151004 awarded by the National Institutes of Health (NIH), and HR0011-17-2-0047 awarded by the Defense Advanced Research Project Agency (DARPA). The government has certain rights in the invention.

### TECHNICAL FIELD

The present invention relates to modified inheritable genetics.

### BACKGROUND

Anopheline mosquitoes are responsible for malaria transmission, with the Anopheles gambiae complex being the most dangerous, contributing to the quarter billion annual Malaria cases¹. Controlling anophelines is an effective strategy to slow disease transmission; however, existing suppression tools such as insecticides and bed nets are becoming increasingly ineffective². Furthermore, plastic mosquito behaviors may be biasing towards increased exophagy³⁻⁵, both contributing to the plateau in the rate of disease reduction and increasing the overall costs of control¹. Therefore, development of more sustainable, efficient, safe, scalable, and cost-effective GM vector control technologies is urgently needed.

Because female mosquitoes spread disease, most vector control campaigns require males to be exclusively released. Producing sufficient males en masse therefore necessitates development of sex-sorting mechanisms in each targeted species, either through mechanical, chemical, or genetic means⁶⁻⁸. Unlike other mosquitoes, sex separation based on pupal size is not possible in A. gambiae^{6,9,10}. Moreover, lines which permit sex sorting via dieldrin resistance no longer exist¹¹, and feeding larvae RNAi suppressing female transcripts yielded incomplete phenotypes¹², suggesting that transgenic methods may be more robust. Directly sorting males is possible by genetically encoding fluorescence on sex chromosomes or near the sex-determinative loci¹³⁻¹⁵, using sex-specific promoters^{16,17}, or using sex-specific alternative splicing^{18,19}. However, these methods require each released male be directly sorted from females at an earlier life stage. For clarity, this is termed a 2:1 sort:ratio; for every two mosquitoes sorted, one male is released. Not only is this labor intensive and somewhat low-throughput, but it requires sorting facilities near release sites, making releases in remote areas exceedingly difficult. Genetic approaches with improved sort:release ratios have been developed which instead genetically kill or incapacitate females^{20,21}. To rear these lines in mass, female-lethal phenotypes are induced through removal of chemical inhibition, or by performing specific genetic crosses^{20,22}. This obviates larval sorting of the released generation providing an order-of-magnitude improvement over a 2:1 sort:release ratio. Unfortunately these technologies have yet to be adapted to Anophelines.

In Anophelines, male-biased or male-sterilizing transgenics have been developed by expressing nucleases targeting the X chromosome during spermatogenesis²³⁻²⁵. However, these transgenes are constitutively dominant complicating mass rearing. Furthermore, these sex-biasing lines cause at most 95% male bias requiring recurrent manual selection. Notwithstanding, African field trials²⁵⁻²⁷ using this system have been conducted, however exhibit low fitness presumably due to low-level transgene expression in other tissues 27 suggesting that an optimal Genetic Sexing Strain (GSS) should not target male-essential factors for inhibition or destruction. Moreover, constitutive transgenic overexpression of the male-determining factor, Yob, causes male-biased sex-distortion in A. gambiae, but it is not fully penetrant and is subject to similar husbandry requirements as those technologies discussed above²⁸. Therefore, while these tools are promising, many experts agree that “currently available technology is not scalable” as a GSS for A. gambiae²⁹. Finally, while sex-distorting gene drives have been developed in A. gambiae^{30,31}, these technologies face political, ethical, and regulatory hurdles prior to release due to the autonomous nature of their spread, making implementation of this technology inappropriate for every application. Furthermore, recent evidence has emerged that these types of suppression drives may be severely curbed by evolution of resistance alleles³².

### SUMMARY OF THE INVENTION

The disclosure provides methods and compositions comprising heritable genetic modifications, and for transgenic animals produced by the methods and systems described herein.

In embodiments, the invention provides a method of suppressing a population of animals, comprising providing a first transgenic animal strain encoding Cas9; providing a second transgenic animal strain encoding gRNA that targets a female essential gene; and sexually crossing the first and second strains to result in both somatic and heritable germline mutations of the female essential gene resulting in daughter killing, and female essential gene mutant males reproductively viable to pass along the female essential gene mutation into subsequent generations, to suppress the population of animals.

In embodiments, the invention provides a transgenic system termed Ifegenia (Inherited Female Elimination by Genetically Encoded Nucleases to Interrupt Alleles) comprising a first transgenic animal strain encoding Cas9 and a second transgenic animal strain encoding gRNA that targets a female essential gene, wherein genetic crossing of the two strains results in both somatic and heritable germline mutations of the female essential gene resulting in daughter killing, and female essential gene mutant males reproductively viable to pass along the female essential gene mutation into subsequent generations. In embodiments, the term Ifegenia can refer to the method of producing the indicated transgenic animals or can refer to the transgenic animals produced by the methods described herein. In particular, Ifegenia can refer to male A. gambiae mosquitoes produced by the methods provided herein, which can preferably include mutant copies of the female essential gene fle, a transgene encoding an endonuclease protein (e.g., Cas9), and a transgene encoding a guide RNA specific for the female essential gene (e.g., fle). The Ifegenia system can also be referred to as Gynecider (GeneticallY eNcodEd CRISPR Induced Daughter ERadicator), which is used herein interchangeably.

In embodiments, the animal is an insect. In embodiments, the insect is a mosquito. In embodiments, the mosquito is A. gambiae. In embodiments, the female essential gene is fle. In embodiments, the invention provides using RNAi, such as described in Krzywinska et al. characterizing the role of the femaleless (fle) gene in sex determination in A. gambiae³³. This technology may be suitable for safe, scalable, confinable, and effective suppression of A. gambiae populations, and is adaptable to other vector species.

In an aspect herein is a transgenic animal, comprising: a first transgene encoding a Cas9 protein; and a second transgene encoding a guide RNA (gRNA) that targets a female essential gene, wherein the first transgene and the second transgene are incorporated into a genome of the transgenic animal, and wherein the second transgene is incorporated into the genome at a location which does not encode the female essential gene. In embodiments, the transgenic animal is a male. In embodiments, the animal is an insect. In embodiments, the insect is a mosquito. In embodiments, the mosquito is A. gambiae. In embodiments, the female essential gene is implicated in regulation of the dsx gene. In embodiments, the female essential gene is fle. In embodiments, the transgenic animal is a male mosquito and the female essential gene is fle.

In embodiments, the transgenic animal is reproductively viable. In embodiments, the presence of the first transgene and the second transgene in a female animal is lethal to the animal. In embodiments, the presence of the first transgene and the second transgene in the female animal is lethal at a larval or pupal stage of development. In embodiments, the first transgene and the second transgene are incorporated at different positions of the genome. In embodiments, the Cas9 protein is a Vasa2-Cas9 protein.

In embodiments, mating of the transgenic animal with a wild type animal produces offspring which contain the first transgene, the second transgene, or both. In embodiments, mating of the transgenic animal with a wild type animal produces offspring which contain a somatic and/or heritable germline mutation of the female essential gene. In embodiments, mating of the transgenic animal with a wild type animal produces offspring which include female essential gene mutant males reproductively viable to pass along the female essential gene mutation into subsequent generations.

In an aspect herein is a method of suppressing a population of animals, comprising releasing a first population of the transgenic animals of provided herein into the population of animals; and allowing the transgenic animals to mate with the population of animals, thereby passing the first transgene and the second transgene into subsequent generations. In embodiments, the method further comprises releasing additional populations of the transgenic animals and allowing the transgenic animals to mate with the population of animals and the subsequent generations, thereby suppressing the population of animals due to the production of non-viable female offspring.

In another aspect herein is a method of preparing a transgenic animal for suppressing a population of animals, comprising providing a first parental transgenic animal strain comprising a transgene encoding a Cas9 protein; providing a second parental transgenic animal strain comprising a second transgene encoding a guide RNA (gRNA) that targets a female essential gene, wherein the second transgene is incorporated into a genome at a position which does not encode the female essential gene; and sexually crossing the first and second parental transgenic animal strains to produce offspring. In embodiments, the offspring include animals which encode the Cas9 protein, the gRNA, or both. In embodiments, the offspring include animals which comprise a somatic and/or heritable germline mutation in the female essential gene. In embodiments, mating of the offspring with a wild type animal passes along a female essential gene mutation in reproductively viable males. In embodiments, the first parental transgenic animal strain is homozygous for the transgene encoding of the Cas9 protein, the second parental transgenic animal strain is homozygous for the transgene encoding of the gRNA, or both are homozygous for their respective transgenes. In embodiments, all or substantially all of the offspring which are viable are males which comprise the transgene encoding the gRNA, the transgene encoding the Cas9 protein, and/or heritable germline mutations in the female essential gene.

Also provided herein in an aspect is a transgenic system for population control of an animal comprising: a first parental transgenic animal strain comprising a first transgene encoding a Cas9 protein; a second parental transgenic animal strain comprising a second transgene encoding a guide RNA (gRNA) that targets a female essential gene, wherein the second transgene is incorporated into a genome at a location which does not encode the female essential gene; wherein genetic crossing of the two strains results in offspring which: include animals which encode the Cas9 protein, the gRNA, or both; include animals which comprise a somatic and/or heritable germline mutation in the female essential gene; and/or include viable males capable of passing along a female essential gene mutation to subsequent offspring. In embodiments, the first parental transgenic animal strain is homozygous for the Cas9 protein and wherein the second parental transgenic animal strain is homozygous for the gRNA.

### BRIEF DESCRIPTION OF THE DRAWINGS

FIG. 1 shows a transgenic crossing system to target fle for mosaic knockout which results in robust female elimination. Panel A shows the gFLE transgene expresses two gRNAs (gRNA7 and gRNA10) targeting fle near the start codon and within the first RNA Recognition Motif (RRM) respectively (gene map). The PolIII U6 promoter facilitates gRNA expression, selection is enabled by Act-EGFP-Sv40, and the transgene was inserted by piggy Bac transgenesis (Terminal Repeats, TR). The Cas9 transgene expresses protein in the adult germline and is maternally deposited into the embryo, and was previously described in Werling et. al.³⁴. Panel B shows individual genotypes are identified by fluorescence with Cas9 marked by 3xP3-DsRed and gFLE marked by Act-GFP. Panel C shows crossing F0 gFLE/+ males to Cas9/+ females yields F1 offspring in Mendelian 1:1:1:1 ratios, with the gFLE/Cas9 female cohort absent. Panel D shows absence of all F1 gFLE/Cas9, and some gFLE/+ female offspring, suggests fle mutagenesis results in female death. This effect is heritable into the F2 and F3 generations with gFLEG/Cas9 males mated to +/+ females able to kill daughters with the appropriate genotypes. Panel E shows crossing homozygous gFLEG/gFLEG males to Cas9/Cas9 females results in complete elimination of genetic daughters from the progeny.

FIG. 2 shows how Ifegenia males can be used for vector control. F0) Mass, factory-based, rearing and sorting of males from a stock FLE/gFLE line, and females from a Cas9/Cas9 line yields a F0 cross from which each fertilized female can produce ˜200 Ifegenia sons during the course of her lifetime. F1) Produced F1 eggs undergo significant Δfle mutagenesis are expected to be 100% Ifegenia males, producing a perfect Genetic Sexing Strain (GSS). Therefore these eggs can be directly released into the environment as part of an Ifegenia multi-generational suppression system (left), or sterilized for release as part of a more traditional SIT-based system (right). Once grown, the resulting F1 males will mate with wild type females to produce their respective population-suppression effects. F2) In a multi-generational suppression system, Ifegenia males remain fertile but ‘load’ Δfle alleles into the population to cause prolonged suppression by daughter killing-providing a non-driving technology. In a traditional SIT-based system involving male sterilization, no F2 offspring would result from released F1 Ifegenia males (right). Due to its profound daughter-killing phenotypes, it has been termed Ifegenia (Inherited Female Elimination by Genetically Encoded Nucleolyticly Interrupted Alleles), in honor of Iphigenia of Greek mythology who was sacrificed by her father, King Agamemnon, to win a great battle.

FIG. 3 shows femaleless knockout females survive embryogenesis. Reported as 1 day old (ldo) larvae counts of the F1 offspring genotypes from a F1 cross of +/gFLE males to +/Cas9 females. Significant embryonic female death would be expected to be observed as an approximate halving of the gFLE/Cas9 group. Panel A shows ldo larvae counts of different genotypes from a +/gFLEG male to +/Cas9 female cross. Two replicates shown stacked in black and grey. Both not significantly different from expected 1:1:1:1 Mendelian ratios, (χ2, p>0.1 and p>0.2 respectively). Panel B shows ldo larvae counts of different genotypes from a +/gFLE_{I} male to +/Cas9 female cross. Two replicates shown stacked in black and grey. Both not significantly different from expected 1:1:1:1 Mendelian ratios, (χ2, p>0.5 and p>0.2 respectively). Panel C shows ldo larvae counts of different genotypes from a +/gFLE_{J} male to +/Cas9 female cross. Two replicates shown stacked in black and grey. Both significantly different from expected 1:1:1:1 Mendelian ratios, (both χ2, p<0.005, consistent with multiple insertions of transgene gFLE_{J}). Family gFLE_{J} was therefore omitted from the analysis in Panel D. Panel D shows data pooled from (A) and (B). Together these results suggest that very slight levels of female embryo lethality are occurring in the gFLE/Cas9 group, however do not account for the bulk of the female-killing phenotype.

FIG. 4 shows larval death assays: femaleless mutants (+/gFLE) and (gFLE/Cas9) die during larvahood. 40 random 1 day old larvae were isolated into separate trays by genotype within 12 h of hatching and reared separately. Individuals were counted and sex-scored upon pupation. Replicate 1 is denoted by triangle, replicate 2 is denoted by a square, replicate 3 is denoted by diamond, and replicate 4 is denoted by circle. For each genotype, the left bar denotes the males counted and the right bar denotes the females counted. Panel A shows mutant femaleless females from family gFLEG were present at 1 do but die during larvahood. Panel B shows mutant femaleless females from family gFLE_{I} were present at ldo but die during larvahood. A third replicate was not performed on this line as the line was deemed sub-optimal for release and omitted from downstream analysis. Panel C shows mutant femaleless females from family gFLE_{J} were present at do1 but die during larvahood.

FIG. 5 shows survival curves for individual replicates of control mosquitoes (ctls 1, 2, and 3) and gFLEG/Cas9 mosquitoes (gFLE (G) reps 1, 2, and 3), with the x-axis showing time (days) and the y-axis showing probability of survival. gFLEG/Cas9 mosquitoes generally showed only modestly reduced survival rates.

FIG. 6 shows male gFLE/Cas9 transheterozygous die only slightly faster than wild type siblings. Panel A shows gFLEG/Cas9 transheterozygous males (n=40) die faster than wild type siblings (N=74) (Log-rank p=0.0017); Panel B shows gFLE_{J}/Cas9 trans heterozygous males (n=105) die slightly faster than wild type siblings (n=79) (Log-rank, p=0.0197) s.

FIG. 7 shows Ifegenia males have high mating competitiveness. Panel A shows offspring genotype ratio from a cross of 35 Ifegenia males to 35 WT females. Genotype ratios were used to calculate the number of WT offspring in Panel B that are attributed to Ifegenia males vs WT males. Among Ifegenia offspring, wild type larvae comprise 34.5% of the brood, and transgenic larvae comprise 65.5% due to transgene linkage on the 2nd chromosome. Panel B shows male mating competition assays of 35 Ifegenia males×35 WT males×35 WT females. All larvae were counted and genotyped. A fraction of WT larvae were attributed to Ifegenia fathers according to the ratios shown in Panel A and reported with transgenic larvae as percent Ifegenia offspring (wt adjusted). Mean and SD shown. Panel C shows egg numbers produced by each replicate. Mean and SD shown, no significant difference unpaired t-test. Panel D shows hatching rate of each replicate. Mean and SD shown, no significant difference, unpaired t-test.

FIG. 8 shows female killing persists in F2 and F3 generations gFLE_{I}/Cas9 and gFLE_{J}/Cas9. In multigenerational experiments following from the crosses outlined in FIG. 1, Panel C, F2 offspring from family gFLE_{I} were followed through the F2 generation, and offspring from family gFLE_{J} were followed through the F3 generation. Phenotypic gFLE/Cas9 females were identified in both families though at significantly reduced frequencies than should be expected by Mendelian segregation.

FIG. 9 shows a model-predicted impact of releases of Ifegenia (1-3 target sites) and pgSIT eggs on A. gambiae population density and elimination. Weekly releases were simulated in a randomly-mixing population of 10,000 adult mosquitoes using the MGDrivE simulation framework⁴⁰ and parameters described in Table 2. Weekly releases of up to 500 Ifegenia or pgSIT eggs per wild adult were simulated over 1-52 weeks. Ifegenia and pgSIT were simulated with cutting rates of 90% per allele, maternal deposition of Cas9 in 90% of embryos when expressed by the mother, and male mating competitiveness of 75% that of wild-type males. For Ifegenia, females homozygous for any Δfle mutant allele were considered unviable, and for pgSIT, males having the system were considered sterile. Panel A shows elimination probability calculated as the percentage of 120 stochastic simulations that result in A. gambiae elimination, depicted for a range of release schemes: 1-52 consecutive weekly releases of 10-500 eggs per adult. Contours partition regions of parameter space that result in a ≥90% elimination probability for each system. Panel B shows Illustrative time-series are depicted for sample releases schemes. Scheme “a” depicts 26 releases of 300 eggs per wild adult, and scheme “b” depicts 26 releases of 100 eggs per wild adult. Panel C shows window of protection is calculated as the percentage of 120 stochastic simulations that result in the A. gambiae population being suppressed by ≥90% for ≥2 years, depicted for 1-52 consecutive weekly releases of 10-500 eggs per adult. Contours partition regions of parameter space that result in a window of protection of ≥2 years for each system. Panel D shows illustrative time-series are depicted for sample releases schemes, with shaded regions representing the window of protection. Scheme “c” depicts 20 releases of 300 eggs per wild adult, and scheme “d” depicts 14 releases of 100 eggs per wild adult

### DETAILED DESCRIPTION

The disclosure provides methods and compositions comprising heritable genetic modifications, and for transgenic animals produced by the methods and systems described herein.

The present disclosure relates, in part, to a method of suppressing a population of animals, comprising providing a first transgenic animal strain encoding Cas9; providing a second transgenic animal strain encoding gRNA that targets a female essential gene; and sexually crossing the first and second strains to result in both somatic and heritable germline mutations of the female essential gene resulting in daughter killing, and female essential gene mutant males reproductively viable to pass along the female essential gene mutation into subsequent generations, to suppress the population of animals.

In embodiments, the invention provides a transgenic system termed Ifegenia (Inherited Female Elimination by Genetically Encoded Nucleases to Interrupt Alleles) comprising a first transgenic animal strain encoding Cas9 and a second transgenic animal strain encoding gRNA that targets a female essential gene, wherein genetic crossing of the two strains results in both somatic and heritable germline mutations of the female essential gene resulting in daughter killing, and female essential gene mutant males reproductively viable to pass along the female essential gene mutation into subsequent generations.

In embodiments, the invention provides animals (e.g., mosquitoes, such as A. gambiae) which contain transgenes encoding a Cas9 protein and gRNA that targets a female essential gene. In embodiments, such animals are releasable into wild populations in order to mate with the wild populations, thereby depositing the transgenes into subsequent populations, as well as mutant copies of the female essential gene. In embodiments, release of such animals results in a reduction or suppression of the wild animal population. In embodiments, the suppression of female animal is particularly reduced or suppressed.

In embodiments, the invention also provides methods of reducing populations of wild animals using transgene containing animals provided herein (e.g., transgenic A. gambiae which comprises transgenes expressing a Cas9 protein and a gRNA targeting a female essential gene). In embodiments, such methods comprise releasing the transgenic animals provided herein and allowing them to mate with animals in a wild population. In embodiments, such methods are effective to pass along the transgenes and mutant genes (e.g., mutant, non-functional copies of the female essential gene) into the wild population. Such methods can be effective to reduce the population by killing of the female animals before maturity in subsequent generations. In embodiments, the methods provided herein provide for iterative releases of the transgene containing animals into the wild population for sustained population reduction and/or suppression.

In embodiments, the invention also provides methods and systems for preparing the transgenic animals provided herein. In embodiments, such methods and systems utilize a first parental transgenic animal strain (e.g., a maternal strain) comprising a transgene expressing a Cas9 protein and a second parental transgenic animal comprising a transgene expressing a guide RNA (gRNA) that targets a female essential gene (e.g., a paternal strain). In embodiments, sexually crossing the two parental transgenic animal strains produces animals which comprise both transgenes (e.g., by utilizing parental strains which are homozygous for the transgenes). In embodiments, the sexual crossing results in only one sex of animal (e.g., males) being produced, and such animals can be used in the methods of population control provided herein. In embodiments, the sexual crossing results in offspring which a) include animals which encode the Cas9 protein, the gRNA, or both; b) include animals which comprise a somatic and/or heritable germline mutation in the female essential gene; and/or c) include viable males capable of passing along a female essential gene mutation to subsequent offspring.

Various further aspects and embodiments of the disclosure are provided by the following description. Before further describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure.

### Transgenic Animals

Provided herein are transgenic animals which can be useful in controlling the population of wild type animals. In embodiments, the transgenic animals can be released into wild type populations, mate with animals of the wild type populations, and have an effect on the genetic makeup of the wild type population of animals which results in a decline in or a suppression of the population of the animal. In embodiments, the transgenic animals contain transgenes capable targeting genes such that offspring of the transgenic animals of one sex are preferentially killed, while offspring of the opposite sex remain viable and able to pass the genes along to subsequent offspring.

In an aspect, provided herein, is a transgenic animal, comprising: one or more transgenes encoding a gene editing system which includes one portion for targeting a female essential gene (e.g., a guide RNA, such as for a CRISPR-Cas9 system) and another portion for performing a gene modification (e.g., inducing a double-stranded break, such as with a Cas9 protein). In embodiments, the one or more transgenes are incorporated into the genome of the animal. In embodiments, the portion targeting the female essential gene is incorporated at a location that does not encode the female essential gene.

In an aspect provided herein is a transgenic animal comprising a first transgene encoding a Cas9 protein; and a second a second transgene encoding a guide RNA (gRNA) that targets a female essential gene. In embodiments, the first transgene and the second transgene are incorporated into a genome of the transgenic animal. In embodiments, the second transgene is incorporated into the genome at a location which does not encode the female essential gene.

In embodiments, the transgenic animal is an insect. In embodiments, the transgenic animal is a pest insect. In embodiments, the insect is an aphid, ant, bee, beetle, cicada, cockroach, cricket, dragonfly, earwig, flea, fly, hornet, locust mantis, moth, mosquito, phasmid, silverfish, termite, tick, or wasp. In preferred embodiments, the insect is a mosquito.

In embodiments, the mosquito is of the Anophelinae or Culicinae subfamily. In embodiments, the mosquito is of the Anophelinae subfamily. In embodiments, the mosquito is of the genus Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina, or Wyeomyia. In embodiments, the mosquito is of the genus Anopheles. In embodiments, the mosquito is of the subgenus Anopheles, Baimaia, cellia, Kerteszia, Lophodomyia, Nyssorhynchus, or Stethomyia. In embodiments, the mosquito is of the subgenus Anopheles. In embodiments, the mosquito A. atroparvus, A. albimanus, A. arabiensis, A. barberi, A. bellator, A. crucians, A. cruzii, A. culicifacies, A. darlingi, A. dirus, A. earlei, A. freeborni, A. funestus, A. gambiae, A. introlatus, A. latens, A. maculipennis, A. moucheti, A. nili, A. punctipennis, A. quadrimaculatus, A. stephensi, A. subpictus, A. sundaicus, or A. walker. In preferred embodiments, the mosquito is A. gambiae.

In embodiments, the transgenic animal can be of a preferred sex. In embodiments, the transgenic animal is a male.

In embodiments, the transgenic animal is reproductively viable. In embodiments, the transgenic animal is able to reproduce (e.g., by mating) with wild type animals of the same type at a rate which is similar to a corresponding wild type animal (e.g., one which does not contain the transgenes). In embodiments, the transgenic animal is able to reproduce at least 25%, at least 35%, at least 40%, at least 50%, at least 60%, at least 70%, at least 75%, at least 80%, at least 85%, or at least 90% as effectively as a corresponding wild type animal. In embodiments, the transgenic animal is able to compete with corresponding wild type animals such that the transgenic animals produce at least 25%, at least 35%, at least 40%, at least 50%, at least 60%, at least 70%, at least 75%, at least 80%, at least 85%, or at least 90% as many offspring as the corresponding wild type animals.

In embodiments, the transgenic animal has a survivability (e.g., average lifespan) which is similar to that of a corresponding wild type animal. In embodiments, the transgenic animal has a survivability (e.g., average lifespan) which is at least 25%, at least 35%, at least 40%, at least 50%, at least 60%, at least 70%, at least 75%, at least 80%, at least 85%, or at least 90% that of a corresponding wild type animal.

In embodiments, the transgenic animal comprises one or more of the transgenes (e.g., the first transgene encoding a Cas9 protein (or other suitable protein) and the second transgene encoding the gRNA (or other suitable targeting means)) is incorporated into the genome of the transgenic animal. In embodiments, the first transgene encoding the Cas9 protein is incorporated into the genome of the transgenic animal. In embodiments, the second transgene encoding the gRNA targeting the female essential gene is incorporated into the genome of the transgenic animal. In embodiments, both the first transgene encoding the Cas9 protein and the second transgene encoding the gRNA targeting the female essential gene are incorporated into the genome of the transgenic animal. In embodiments, both the first transgene and the second transgene are incorporated into the same position of the genome (e.g., under control of the same operable promotor sequence, or under control of different operative promoter sequences but adjacent or substantially adjacent to each other). In preferred embodiments, the first transgene and the second transgene are incorporated at different positions of the genome.

In embodiments wherein the transgene(s) are incorporated into the genome, the transgene(s) can be incorporated into a targeted site (e.g., at a desired location within the genome). In embodiments, the transgene(s) are incorporated at a position other than that of the female essential gene. In embodiments, the transgene(s) are incorporated at a position which is sufficiently far from the female essential gene such that the transgene(s) will not be copied into the female essential gene.

In embodiments, it is preferred that the transgene(s) do not act as a gene drive (e.g., self-propagate within the genome such that transgene(s) are passed down to offspring at super-Mendelian rates). In such embodiments, the transgene(s) (e.g., the transgene encoding the gRNA targeting the female essential gene and/or the transgene encoding the Cas9 protein) are placed within the genome at a location such that when the gene editing system targets the female essential gene, the transgene(s) are not incorporated into the female essential gene. In embodiments, the transgenes are passed down to offspring at substantially standard Mendelian frequency.

In embodiments, the transgene(s) (e.g., the transgene encoding the gRNA targeting the female essential gene and/or the transgene encoding the Cas9 protein) are incorporated at a site which is at least 1000, 2000, 3000, 4000, 5000, or 10000 nucleotides away from the female essential gene. In embodiments, the transgene(s) (e.g., the transgene encoding the gRNA targeting the female essential gene and/or the transgene encoding the Cas9 protein) are incorporated at least 1000, 2000, 3000, 4000, 5000, or 10000 nucleotides away from the portion of the female essential gene targeted by the gRNA. In embodiments, the transgene(s) (e.g., the transgene encoding the gRNA targeting the female essential gene and/or the transgene encoding the Cas9 protein) are incorporated on a different chromosome than the female essential gene. In embodiments, the transgene(s) (e.g., the transgene encoding the gRNA targeting the female essential gene and/or the transgene encoding the Cas9 protein) are incorporated on a different chromosome arm than the female essential gene.

In embodiments wherein the transgenic animal is a mosquito, the animal can have the transgene(s) (e.g., the transgene encoding the gRNA targeting the female essential gene and/or the transgene encoding the Cas9 protein) incorporated at a number of suitable positions. In embodiments, the transgene(s) are incorporated on chromosome 2. In embodiments, the transgene(s) are incorporated at chromosome 2 L and/or chromosome 2R. In embodiments, the transgene encoding the gRNA and the transgene encoding the Cas9 protein are incorporated on chromosome 2. In embodiments, the one of the transgene encoding the gRNA and the transgene encoding the Cas9 protein is incorporated at chromosome 2 L and the other is incorporated at chromosome 2R. In embodiments, the transgene encoding the gRNA is incorporated at chromosome 2R. In embodiments, the transgene encoding the Cas9 protein is incorporated at chromosome 2 L.

In embodiments wherein the transgenic animal contains multiple transgenes on the same chromosome (e.g., a transgenic mosquito having a transgene encoding a Cas9 protein at chromosome 2 L and a transgene encoding a gRNA at chromosome 2R), each of the transgenes can be either on the same homolog (e.g., both transgenes are present on the paternal homolog) or the transgenes can be present on opposite homologs (e.g., one transgene, such as the Cas9 protein encoding transgene, is present on the maternal homolog and the another transgene, such as the one encoding the gRNA, is present on the paternal homolog). In embodiments, the transgenic animal is descended from maternal and paternal transgenic animals each of which comprise one of either the transgene encoding the Cas9 protein or the transgene encoding the gRNA on the same chromosome (e.g., the maternal parental transgenic animal comprises the transgene encoding the gRNA at chromosome 2R and the maternal parental animal comprises the transgene encoding the gRNA at chromosome 2 L). In such cases, the transgenic animal is expected to have the transgene encoding the Cas9 protein and the transgene encoding the gRNA present on opposite homologs. However, as is apparent to a skilled artisan, owing to chromosomal crossover, a certain number of such transgenic animals will comprise the two transgenes on the same homolog (and thus such animals would pass on both transgenes to subsequent generations together).

In embodiments, the transgene(s) include additional elements (e.g., promoters, enhancers, selection markers, reporter elements, etc.) which allow for maximal utility and optimal expression of the key genes of the transgenes. In embodiments, the transgene(s) include a promotor element (e.g., U6 promoter, Act5C promoter, 3xP3, vasa2, etc.). In embodiments, the transgene(s) include an enhancer element (e.g., Sv40, etc.). In embodiments, the transgenes include a reporter element (e.g., a fluorescent protein such as green fluorescent protein (GFP), dsRed, etc.). In embodiments, the reporter element is used as a selection marker in order to select for transgenic animals which contain one or more of the desired transgenes (e.g., for purposes of identifying animals which possess the desired transgene, such as for sorting purposes).

In embodiments, the transgenic animal comprises a first transgene encoding a Cas9 protein. In embodiments, the transgene encoding the Cas9 protein comprises one or more promoters which enhances germline mutagenesis. In embodiments, the transgene encoding the Cas9 protein includes a Vasa2 promoter. In embodiments, the transgene is a Vasa2-Cas9.

In embodiments, alternatives to the Cas9 protein can be used in order to effectuate the required mutagenesis of the target female essential gene. In embodiments, an alternative endonuclease is used instead of Cas9. Exemplary endonucleases which can be used include meganucleases, Transcription Activator Like Effector Nucleases (TALEN), a Zinc-Finger Nucleases (ZFN), and other Clustered Regularly Interspaced Short Palindromic Repeats (CRISPR)-associated systems (Cas), and derivatives thereof. In embodiments, the transgenic animal comprises a transgene encoding one of these endonucleases. In embodiments, the endonuclease is one which can be targeted to a desired endonuclease site with a guide nucleic acid (e.g., a gRNA).

In embodiments, the transgenic animal comprises a second transgene encoding a gRNA targeting a female essential gene. In embodiments, the transgenic animal comprises a second transgene encoding a plurality (e.g., 2, 3, 4, or more) of gRNAs targeting the female essential gene. In embodiments, the transgenic animal comprises multiple transgenes encoding a plurality of gRNAs (e.g., multiple transgenes each encoding a different gRNA). In embodiments, the transgenic animal comprises a second transgene encoding two gRNAs, each targeting a different portion of the female essential gene.

In embodiments, the transgenic animal comprises a transgene encoding a gRNA targeting a female essential gene. A female essential gene is a gene which, when mutated or otherwise made non-functional, has a substantially more deleterious effect on female animals than males. These effects can include death of the female animals or other developmental abnormalities. In preferred embodiments, elimination of functional copies of the female essential gene causes death of the female animals, more preferably at an early stage of life (e.g., before sexual maturity, preferably before maturity into an adult (e.g., death at a pupal or larval stage in instances wherein the transgenic animal is an insect). In embodiments, elimination of both functional copies of the female essential gene causes death in at least 95%, 96%, 97%, 98%, 99%, or in 100% of the female animals before they reach adulthood.

In embodiments wherein the transgenic animal is a mosquito (e.g., A. gambiae), the transgenic animal can comprise a gRNA targeting a female essential gene. In embodiments, the female essential gene is implicated in the development of female mosquitos. In embodiments, the female essential gene is implicated in the differentiation of female mosquitoes from male mosquitos. In embodiments, the female essential gene is one in the doublesex (dsx) gene pathway. In embodiments, the female essential gene is implicated in regulation of the dsx gene. In embodiments, the female essential gene is a regulator of the dsx gene. In embodiments, the female essential gene regulates splicing of the dsx gene. In embodiments, the female essential gene is the femaleless (fle) gene.

In embodiments, the transgene encoding the gRNA targets the fle gene. In embodiments, the gRNA targets an exon of the fle gene. In embodiments, the gRNA targets the first exon of the fle gene. In embodiments, the transgene encodes multiple gRNAs which target the fle gene. In embodiments, the transgene encodes two gRNAs which target the fle gene. In embodiments, both of the gRNAs target exons of the fle gene. In embodiments, both gRNAs target the first exon of the fle gene. In embodiments, the gRNAs target different exons of the fle gene.

In embodiments, introduction of mutations in the fle gene as a result of the gRNA targeting of the fle gene is lethal to female animals (i.e., female mosquitos). In embodiments, loss of function of the fle gene is lethal to the female animals. In embodiments, loss of function of the fle gene is lethal to the female animals at an early stage of development. In embodiments, loss of function of the fle gene is lethal to the female animals at the larval or pupal stage of development. In embodiments, loss of function of the fle gene is lethal to the female animals at the larval or pupal stage, or earlier in development.

In embodiments, the presence of the transgene encoding a Cas9 protein (or other suitable endonuclease provided herein) and the presence of a gRNA targeting fle in a female animal (i.e., a female mosquito) is lethal to the animal. In embodiments, the presence of the two transgenes inherited from one or both parents in a female animal is lethal to the animal. In embodiments, the presence of the transgene encoding the gRNA alone is sufficient to kill the female animal, owing either to parental (e.g., maternal) deposition of sufficient quantity of endonuclease (e.g., Cas9) to enable modification of the fle gene, or potentially to a functioning of the gRNA by another mechanism (e.g., such as by acting as small interfering RNA or another RNA silencing mechanism). In embodiments, the presence of the two transgenes in the female animal is lethal at a larval or pupal stage of development. In embodiments, the presence of the two transgenes in the female animal is lethal at a larval or pupal stage of development, or at an earlier stage of development. In embodiments, the presence of both transgenes is lethal to at least 95%, 96%, 97%, 98%, 99%, or to 100% of the female animals before they reach adulthood.

In embodiments, the presence of the transgene encoding a Cas9 protein (or other suitable endonuclease provided herein) and the presence of a gRNA targeting fle in a male animal (i.e., a male mosquito) is non-lethal to the animal. In embodiments, the presence of both transgenes has minimal or no effect on the viability or lifespan of the male animal. In embodiments, the presence of both transgenes has minimal or no effect on the ability of the male animal to mate. In embodiments, the presence of both transgenes introduces somatic and/or germline mutations into the fle gene of the male animal. In embodiments, the presence of both transgenes introduces somatic and/or germline mutations into both alleles of the fle gene of the male animal. In embodiments, the presence of both transgenes introduces germline mutations into both alleles of the fle gene of the male animal. In embodiments, the presence of both transgenes allows the male animal to pass along mutant copies of the fle gene to offspring, even in offspring which do not receive one or both transgenes.

In embodiments, mating of the transgenic animal with a wild type animal is capable of producing offspring. In embodiments, the offspring will contain one or more of the transgenes provided herein or a mutant copy of a female essential gene as provided herein, or one or more of the transgenes and a mutant copy of a female essential gene as provided herein. In embodiments, mating of the transgenic animal with a wild type animal produces offspring which contain the first transgene, the second transgene, or both. In embodiments, mating of the transgenic animal with a wild type animal produces offspring which contain a somatic and/or heritable germline mutation of the female essential gene. In embodiments, mating of the transgenic animal with a wild type animal produces offspring which contain a somatic mutation of the female essential gene. In embodiments, mating of the transgenic animal with a wild type animal produces offspring which contain a germline mutation of the female essential gene. In embodiments, mating of the transgenic animal with a wild type animal produces offspring which contain germline mutations and somatic mutations of the female essential gene. In embodiments, mating of the transgenic animal with a wild type animal produces offspring which contain germline mutations and somatic mutations of the female essential gene even without the presence of one or both of the transgenes in the offspring. In embodiments, mating of the transgenic animal with a wild type animal produces offspring which include female essential gene mutant males reproductively viable to pass along the female essential gene mutation into subsequent generations.

The transgenic animals can be at any stage of development. In embodiments, the transgenic animals are adults, pupae, larvae, or eggs. In embodiments, the transgenic animals are provided as fertilized eggs. In embodiments, the transgenic animals are provided as adults. In embodiments, the transgenic animals are provided as a population of fertilized eggs. In embodiments, the transgenic animals are provided as a population of fertilized eggs which may be interspersed with unfertilized eggs or eggs fertilized with other animals (e.g., non-transgenic animals of the instant disclosure may be interspersed within a population of fertilized eggs which comprise the transgenic animals of the instant disclosure).

### Methods for Preparing Transgenic Animals

Also provided herein are methods of preparing the transgenic animals (e.g., mosquitoes) described herein. In embodiments, the methods comprise providing parental transgenic animals which comprises transgenes encoding one or more portions of the transgenes necessary for the transgenic animals of the disclosure to suppress populations according to the methods provided herein. In embodiments, the methods comprise sexual crossing parental strains such that one or more offspring of the sexual cross comprises all of the desired transgenes. Identification of such offspring can be accomplished by sorting the offspring (e.g., but measuring or detecting the presence of reporter genes in the offspring which are indicative of the presence of the transgenes) or can be accomplished by designing the parental transgenic animals such that all offspring should contain the desired transgenes (e.g., by providing homozygous parental transgene animals). In preferred embodiments, no sorting is required to identify the offspring containing the desired transgenes (e.g., only desired offspring will result from the terminal cross such that the offspring (e.g., fertilized eggs) can be selected directly therefrom without sorting).

In an aspect herein is a method of preparing a transgenic animal (e.g., an insect such as a mosquito) for suppressing a population of animals, comprising: providing a first parental transgenic animal strain encoding an endonuclease protein (e.g., a Cas9 protein or other endonuclease provided herein); providing a second parental transgenic animal strain encoding a guide RNA (gRNA) that targets a female essential gene; and sexually crossing the first and second parental transgenic animal strains to produce offspring. In embodiments, the offspring comprise the transgenic animal. In embodiments, the offspring comprises individuals which contain genes (i.e., transgenes) which encode the endonuclease protein and/or the gRNA, thereby providing individual animals which can be used to suppress an animal population.

In embodiments, at least one of the parental transgenic animal strains is homozygous for the transgene. Having a parental transgenic animal strain be homozygous for one of the transgenes substantially ensures that the offspring possess at least one copy of the relevant transgene, thereby eliminating or minimizing the need for sorting of the offspring to identify individuals with the desired transgene. In embodiments, the first parental transgenic animal strain encoding the endonuclease protein (e.g., the Cas9 protein) is homozygous for the endonuclease protein transgene. In embodiments, the second parental transgenic animal strain encoding the gRNA is homozygous for the transgene encoding the gRNA. In embodiments, both the first parental transgenic animal strain encoding the endonuclease protein (e.g., the Cas9 protein) is homozygous for the endonuclease protein transgene and the second parental transgenic animal strain encoding the gRNA is homozygous for the transgene encoding the gRNA. In such embodiments, sorting the offspring to identify animals having desired properties (e.g., identifying male animals comprising both transgenes) is not required because the presence of both transgenes is lethal to female animals (thereby eliminating the need to sex sort) and because the offspring males are ensured to have both transgenes due to homozygous parental transgenic animal strains.

In embodiments, it is preferable that a particular sex of parent encode the endonuclease protein (e.g., the Cas9 protein) or the gRNA. In embodiments, the maternal parental transgenic animal encodes the endonuclease protein (e.g., the Cas9 protein). In embodiments, the paternal transgenic animal strain encodes the gRNA.

In embodiments, providing parental transgenic animal strains (e.g., a parental transgenic animal strain encoding an endonuclease protein such as Cas9 and/or a parental transgenic animal strain encoding a gRNA that targets a female essential gene) comprises manufacturing the parental transgenic animal strains. In embodiments, providing parental transgenic animal strains comprises introducing the relevant genetic material (e.g., the gene encoding the endonuclease protein or the sequence encoding the gRNA) in a wild type organism. Methods of introducing such genetic material are well known in the art. In embodiments, introducing the relevant genetic material comprises incorporating the genetic material into the genome of the organism as an embryo. After identification or confirmation of embryos or further developed animals (e.g., adults, pupae, larvae, etc.) which contain the incorporated genetic material, such animals can either be used directly to prepare the desired transgenic animals (e.g., by sexually crossing the animals as the transgenic parental strain) or such animals can be outcrossed with wild type animals to prepare families of animals which contain the desired genetic material. Such outcrossed families can be maintained and sexually crossed in a variety of manners in order to maintain stock having the desired genetic material with the desired genetics (e.g., maintaining stock of animals which are homozygous for the desired genetic material or heterozygous for the desired genetic material). Such methods can comprise manual sexing of precursor strains to select individual animals which have the desired traits for subsequent processing. From the outcrossed families, animals comprising the desired genetic material can sorted and identified in order for future sexual crosses in order to maintain desired stock.

In embodiments, offspring produced by the methods provided herein include animals which contain one or more of the transgenes of the parental transgenic animal strains. In embodiments, the offspring include animals which encode the endonuclease protein (e.g., the Cas9 protein), animals which encode the gRNA that targets the female essential gene (e.g., a guide RNA targeting fle), or both. In embodiments, the offspring include animals which encode the endonuclease protein (e.g., the Cas9 protein). In embodiments, the offspring include animals which encode the gRNA that targets the female essential gene (e.g., a guide RNA targeting fle). In embodiments, the offspring include animals which encode both the endonuclease protein and the gRNA. In embodiments, the offspring include animals which comprise the transgene which encodes the endonuclease protein, animals which comprise the transgene which encodes the gRNA that targets the female essential gene, or both. In embodiments, the offspring comprise animals which comprise the transgene which encodes the endonuclease protein. In embodiments, the offspring comprise the transgene which encodes the gRNA that targets the female essential gene. In embodiments, the offspring include animals which comprise both the transgene which encodes the endonuclease protein and the transgene which encodes the gRNA that targets the female essential gene. In embodiments, at least 50%, 60%, 70%, 80%, 90%, or 95% of the offspring comprise the transgene which encodes the endonuclease and the transgene which encodes the gRNA. In embodiments, substantially all of the offspring comprise both transgenes.

In embodiments, the offspring include animals which comprises a somatic and/or heritable germline mutation in the female essential gene. In embodiments, the offspring animals which comprise both the transgene which encodes the gRNA and the transgene which encodes the endonuclease (e.g., the Cas9 protein) contain somatic and heritable germline mutations in the female essential gene owing to the action of the two transgenes during the embryonic stage. In embodiments, offspring which include only the gRNA transgene will also contain somatic and heritable germline mutation in the female essential gene owing to expression of the gRNA and its interaction with Cas9 deposited in the embryo from one if the parental transgenic animal strains. In embodiments, the offspring include animals which comprises both somatic and heritable germline mutations in the female essential gene. In embodiments, at least 50%, 60%, 70%, 80%, 90%, or 95% of the offspring animals comprise both somatic and heritable germline mutations in the female essential gene.

In embodiments, the offspring include reproductively viable male animals. In embodiments, at least 50%, 60%, 70%, 80%, 90%, or 95% of the viable offspring are reproductively viable males. In embodiments, the viable offspring consist essentially of reproductively viable males. In embodiments, all or substantially all of the offspring which are viable are males which comprise the transgene encoding the gRNA, the transgene encoding the Cas9 protein, and/or heritable germline mutations in the female essential gene. In embodiments, mating of reproductively viable male offspring from the parental transgenic animal strains with wild type animals passes along a female essential gene mutation to a successive generation. In embodiments, mating of reproductively viable male offspring from the parental transgenic animal strains with wild type animals passes along at least one of the transgenes to a successive generation. In embodiments, mating of the offspring with wild type animals distributes female essential gene mutations and the transgenes into the population. In such embodiments, release of the transgenic animals loads these genes into the wild type population. In embodiments, this distribution of female essential gene mutations and transgenes into the population of wild type animals assists in sustained population reduction or suppression upon successive releases of offspring of the parental transgenic animal strains as further offspring will be more likely to result in the desired daughter-killing effects.

In embodiments, the method of preparing the transgenic animal for suppressing the population of animals produces a population of offspring (e.g., reproductively viable male offspring which comprise both transgenes) which can be readily deployed into a population suppression strategy without the need for sorting. In embodiments, the parental transgenic animal strains are configured such that all offspring have the desired transgenes and sex. In embodiments, the parental transgenic animal strains are configured such that all viable offspring have the desired transgenes and sex. In embodiments, the parental transgenic animal strains are configured such that all viable offspring are males which comprises both desired transgenes (e.g., the endonuclease such as the Cas9 protein and the gRNA that targets the female essential gene). In such a configuration, no sorting of the animals desired to be released for suppression of the population is required.

While embodiments which do not require sorting of offspring for release into the wild type population are preferred owing to their streamlined approach and enhanced efficiency, in embodiments, the method of preparing the transgenic animals can comprise a step of sorting offspring to identify offspring having the desired traits (e.g., male animals which comprise the transgene encoding the endonuclease protein and the transgene encoding the gRNA). The step of sorting the transgenic animals to identify the offspring with the desired traits can be performed where homozygous parental transgenic animal strains are not available. In embodiments, it may also be acceptable to release populations of animals which include those of the desired genotype along with other animals which do not possess the desired genotype.

In embodiments, the methods provided herein are performed in a laboratory or factory setting at or near a desired release location of the animals. In embodiments, the laboratory or factory maintains the stock of parental transgenic animal strains in order to facilitate iterative releases of transgenic animals. In embodiments, the laboratory or factory includes all necessary equipment for storage, maintenance, sorting, distribution (e.g., field release of animals), crossing, and rearing of the animals. In embodiments, the laboratory or factory is provided within a distance of 20 miles, 30 miles, 40 miles, 50 miles, 75 miles, 100 miles, or 200 miles of the desired release location.

### Systems of and for Preparing Transgenic Animals

Also provided herein are systems (e.g., combinations of tools, animals, and other components) which are useful in the preparation of transgenic animals as provided herein, useful in methods of population control as provided herein, or otherwise useful for executing and practicing the invention as provided herein. In embodiments, provided herein are transgenic systems (e.g., sets of transgenic animal strains, such as parental strains, precursors thereof, and/or transgenes (and, optionally, associated reagents, tools, kits, etc. which can be used to insert said transgenes into an organism)) which can be used to prepare transgenic animals according to the instant disclosure. In an aspect, provided herein, is a transgenic system which is configured to prepare a transgenic animal as described herein.

In embodiments herein is a transgenic system comprising a first parental transgenic animal strain encoding an endonuclease protein (e.g., a Cas9 protein); and a second parental transgenic animal strain encoding a guide RNA (gRNA) that targets a female essential gene. In embodiments, crossing of the two strains results in offspring. In embodiments, the offspring include animals which encode the endonuclease protein, the gRNA, or both. In embodiments, the offspring include animals which comprise a somatic and/or heritable germline mutation in the female essential gene. In embodiments, the offspring include viable males capable of passing along a female essential gene mutation to subsequent offspring.

In embodiments, the system comprises a first parental transgenic animal strain encoding an endonuclease protein. In embodiments, the first parental transgenic animal strain encodes a Cas9 protein. In embodiments, the Cas9 protein is the Vasa2-Cas9 protein. In embodiments, the transgene encoding the endonuclease protein is incorporated into the genome of the first parental transgenic animal strain. In embodiments, the transgene encoding the endonuclease protein is incorporated into the genome at a position which does not encode (or is not near) the female essential gene. In embodiments, the first parental transgenic animal strain is homozygous for the endonuclease protein. In embodiments, the first parental animal strain comprises homozygous female animals. In embodiments, the system uses (e.g., use in preparing transgenic animals for use in methods of population control as provided herein) only female transgenic animals encoding the endonuclease protein. In embodiments, the system uses (e.g., use in preparing transgenic animals for use in methods of population control as provided herein) only female transgenic animals which are homozygous for the endonuclease protein.

In embodiments, the system also comprises additional members of the first parental transgenic animal strain useful in maintaining the population of desired parental transgenic animals for use in methods of preparing the desired transgenic animals. For example, the system can comprise animals from the first parental transgenic animal strain homozygous for the endonuclease protein for use in crossing with the second parental transgenic strain and animals which are heterozygous for the endonuclease protein. Animals heterozygous for the endonuclease protein can be used to cross with other animals which encode the endonuclease protein in order to expand the genetic stock as well as prepare additional homozygous animals (e.g., by crossing with other individuals and sorting for homozygous individuals). Similarly, wild type animals can also be incorporated into the system for further genetic diversification followed by appropriate sorting to eventually arrive at homozygous individuals.

In embodiments, the system comprises a second parental transgenic animal strain encoding a gRNA targeting a female essential gene (e.g., fle). In embodiments, the second parental transgenic animal strain encodes a plurality of such gRNAs. In embodiments, the transgene encoding the gRNA is incorporated into the genome of the second parental transgenic animal strain. In embodiments, the transgene encoding the gRNA is incorporated into the genome at a position which does not encode (or is not near) the female essential gene. In embodiments, the second parental transgenic animal strain is homozygous for the gRNA. In embodiments, the second parental animal strain comprises homozygous male animals. In embodiments, the system uses (e.g., use in preparing transgenic animals for use in methods of population control as provided herein) only male transgenic animals encoding the gRNA. In embodiments, the system uses (e.g., use in preparing transgenic animals for use in methods of population control as provided herein) only male transgenic animals which are homozygous for the gRNA.

In embodiments, the system also comprises additional members of the second parental transgenic animal strain useful in maintaining the population of desired parental transgenic animals for use in methods of preparing the desired transgenic animals. For example, the system can comprise animals from the second parental transgenic animal strain homozygous for the gRNA for use in crossing with the second parental transgenic strain and animals which are heterozygous for the gRNA. Animals heterozygous for the gRNA can be used to cross with other animals which encode the endonuclease protein in order to expand the genetic stock as well as prepare additional homozygous animals (e.g., by crossing with other individuals and sorting for homozygous individuals). Similarly, wild type animals can also be incorporated into the system for further genetic diversification followed by appropriate sorting to eventually arrive at homozygous individuals.

In embodiments, it is helpful and desired that the two parental transgenic animal strains be maintained in a segregated manner in order to ensure that the transgenes are not crossed except under controlled circumstances in which it is desirous to produce the transgenic animals useful in suppression of population control (e.g., the animals which comprises a transgene encoding the gRNA targeting the female essential gene and a transgene encoding the endonuclease protein, such as Ifegenia males as described herein). In embodiments, neither of the first transgenic animal strain or the second transgenic animal strain encodes the transgene of the other transgenic animal strain (e.g., the first transgenic animal strain encoding the endonuclease protein does not also encode the gRNA and the second transgenic animal strain encoding the gRNA does not also encode the endonuclease protein).

In embodiments, the transgenic system is configured such that crossing of animals of the first parental transgenic animal strain (i.e., the animal strain which encodes the endonuclease protein) with animals of the second parental transgenic animal strain (i.e., the animal strain which encodes the gRNA) produces offspring transgenic animals with desired properties (e.g., as in any of the transgenic animals described herein which can be useful in methods of population control). In embodiments, the offspring transgenic animals include animals with one or more of the following properties: the animals encode the endonuclease protein, the gRNA, or both; the animals comprise a somatic and/or heritably germline mutation in the female essential gene; and/or the animals are viable males capable of passing along a female essential gene mutation (e.g., a mutation which inactivates the gene) to subsequent offspring. In embodiments, the offspring include animals which have two of these properties.

In embodiments, the offspring include animals which have each of these properties.

In embodiments, the offspring transgenic animals produced by a transgenic system as provided herein include animals which encoding the endonuclease protein, the gRNA, or both. In embodiments, the offspring include animals which encode both the endonuclease protein (e.g., Cas9) and the gRNA. In embodiments, the offspring transgenic animals produced by a transgenic system as provided herein include animals which comprise a somatic and/or heritable germline mutation in the female essential gene. In embodiments, the offspring transgenic animals produced by a transgenic system as provided herein include animals which comprise a somatic mutation in the female essential gene. In embodiments, the offspring transgenic animals produced by a transgenic system as provided herein include animals which comprise a heritable germline mutation in the female essential gene. In embodiments, the offspring transgenic animals produced by a transgenic system as provided herein include animals which comprise a somatic and heritable germline mutation in the female essential gene. In embodiments, the offspring transgenic animals produced by a transgenic system as provided herein include viable males capable of passing along a female essential gene mutation to subsequent offspring.

In embodiments, the transgenic system provided herein produces no viable female offspring. In embodiments, crossing of the first transgenic animal strain with the second transgenic animal strain produces no viable female offspring. In embodiments, the viable offspring produced by the transgenic system consists essentially of males (i.e., no genotypic female animals survive to adulthood/maturity). In embodiments, crossing of the first transgenic animal strain with the second transgenic animal strain produces essentially only viable males (i.e., no genotypic female animals survive to adulthood/maturity).

### Methods of Population Control

Also provided herein are methods of controlling (e.g., reducing or suppressing) populations of animals using the methods, animals, and systems provided herein. In embodiments, the methods of population control include releasing transgenic animals (e.g., mosquitoes) of the instant disclosure (e.g., Ifegenia male mosquitoes) into the environment such that the animals will mate with animals in the wild population. In embodiments, mating of the transgenic animals with wild type animals in the wild population introduces transgenes and/or mutant (e.g., non-functional) version of a female essential gene into the wild type populations. In embodiments, successive generations see reduced populations owing to these being distributed throughout the population. In embodiments, these genes are pass to subsequent generations at standard Mendelian frequency (i.e., the genes are not part of gene drive elements). In embodiments, the methods of controlling population are thus temporal in nature and can be halted or phased out at an appropriate time without causing substantial long lasting changes to the genotype of the wild type populations. In embodiments, the control of the population requires iterative distributions of transgenic animals to keep the population controlling genes in circulation among the wild population. In embodiments, the suppression or reducing of the population can readily be stopped by simply stopping subsequent releases of the transgenic animals.

In an aspect described herein is a method of suppressing a population of animals, In embodiments, the method comprises releasing a first population of the transgenic animals as described herein (e.g., Ifegenia male mosquitoes) into the population of animals. In embodiments, the method comprises allowing the transgenic animals to mate with the population of animals. In embodiments, the transgenes of the transgenic animals are passed into subsequent generations. In embodiments, modified genes of the transgenic animals (e.g., mutant female essential genes) are passed into subsequent generation.

In embodiments, the transgenic animal used in a method of suppressing a population of animals as described herein can be any of the animals provided herein. In embodiments, the transgenic animal is an insect. In embodiments, the transgenic animal is an mosquito. In embodiments, the transgenic animal is an A. Gambiae. In embodiments, the transgenic animal is an Ifegenia mosquito as provided herein. In embodiments, the animals are of the same type as the transgenic animals (e.g., the transgenic animals are derived from the same species of animal which is targeted for population suppression).

In embodiments, the method comprises releasing a population of the transgenic animals into a population of animals (e.g., a wild population of the animals). In embodiments, releasing the population of animals comprises depositing the transgenic animals into an environment where they are likely to encounter the population. In embodiments, releasing the population of transgenic animals comprises depositing the transgenic animals into an environment at a desired stage of development. In embodiments, the population of transgenic animals is deposited into the environment as an adult, juvenile, or an earlier stage of development. In embodiments, the population of transgenic animals is deposited into the environment as fertilized eggs (e.g., fertilized mosquito eggs). In embodiments, the population of transgenic animal is deposited into the environment in a manner mimics the way in which wild type animals develop (e.g., fertilized mosquito eggs are placed at a location where one would expect to find wild mosquito eggs).

In embodiments, the size of the population of transgenic animals released into the environment is selected to impart a desired effect on the wild population. For example, in embodiments in which the target animal population is mosquitos, it may be desirable to release a high number of fertilized eggs relative to the number of animals in the wild population (e.g., at least 100 eggs, 200 eggs, 300 eggs, 400 eggs, or 500 eggs per adult mosquito in the wild population or wild area). The selection of the number of transgenic animals released will depend on the animal and a variety of factors, such as the ability of the transgenic animals to compete with wild type animals for reproduction, likelihood of survival of the released animals (or likelihood of reaching adulthood), lifespan of released animals, how frequently animals will be released, and other such factors. In embodiments, it can be desirable to increase the number of animals released over time in order to monitor the effects before larger releases occur.

In embodiments, the method of suppressing the population of animals with the transgenic animals provided herein comprises iterative releases of the animals. In embodiments, iterative releases of the transgenic animals are more effective for reducing the population of the animals as the transgenes and mutant copies of the female essential gene are more widely distributed throughout the population. In embodiments, the method of suppressing the population of animals comprising releasing additional populations of the transgenic animals and allowing the transgenic animals to mate with the population of animals and the subsequent generations, thereby suppressing the population of animals due to the production of non-viable female offspring. In embodiments, iterative releases of the transgenic animals can be release of the same amount of transgenic animals or can be different amounts.

In embodiments, the method of suppressing the population of animals comprises iterative releases of the transgenic animals over a period of time. In embodiments, further populations of the transgenic animals are released over a period of at least 1 month, at least 2 months, at least 3 months, at least 6 months, at least 1 year, at least 1.5 years, at least 2 years, at least 2.5 years, at least 3 years, at least 3.5 years, at least 4 years, at least 4.5 years, or at least 5 years. In embodiments, further populations are released or anticipated to be released for an indefinite period of time.

In embodiments, further populations of the transgenic animals are released according to a set schedule. In embodiments, further populations of the transgenic animals are released, for example, daily, twice-weekly, three time a week, weekly, every 10 days, every two weeks, every three weeks, every four weeks, monthly, bi-monthly, every six weeks, every eight weeks, every two months, every three months, or at any appropriate interval.

In embodiments, the release of the transgenic animals is targeted to reduce the wild population of animals to an indicated threshold. In embodiments, the release of the transgenic animals is targeted to and effective to reduce the population of wild type animals by at least, for example, 25%, 30%, 35%, 40%, 45%, 50%, 55%, 60%, 65%, 70%, 75%, 80%, 85%, or 90% compared to the population before population suppression measures are taken.

In embodiments, the methods of population control provided herein can be utilized alongside other methods known in the art for controlling the population of animals. Such methods can include without limitation pesticide based methods, Sterile Insect Techniques (SITs, such as precision guided SITs (pgSIT)), or other methods.

### Certain Definitions

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (MJ. Gait, ed., 1984); Methods in Molecular Biology, Humana Press; Cell Biology: A Laboratory Notebook (J. E. Cellis, ed., 1998) Academic Press; Animal Cell Culture (R.I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and CC. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (CA. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J. B. Lippincott Company, 1993). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a transgenic cell and/or a method that “comprises” a list of elements (e.g., components, features, or steps) is not necessarily limited to only those elements (or components or steps), but may include other elements (or components or steps) not expressly listed or inherent to the transgenic cell and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment.

Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. It is understood that aspects and embodiments of the invention described herein include “consisting” and/or “consisting essentially of” aspects and embodiments.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

It will be further understood that there are a number of values disclosed therein, and

that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length=15%, +10%, +9%, +8%, +7%, +6%, +5%, +4%, +3%, +2%, or +1% about a reference quantity, level, value, number, frequency, percentage, dimension, size, amount, weight or length.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The section headings used herein are for organizational purposes only and are not to be construed as limiting the subject matter described.

As used herein, “transgenic”, “engineered” or “genetically modified” or “transformed” are used interchangeably, wherein a cell has been manipulated by means of molecular reprogramming of a genomic sequence (e.g. by insertion, deletion, or substitution). Said cells include the somatic and germ cells and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein.

As used herein, “inactivating mutations” are used to describe mutations, i.e., insertions, deletions, or substitutions, of genomic nucleic acids that result in lack of formation of a transcript or translated product compared to that of a wild type, or naturally occurring, genomic nucleic acid sequence. An inactivating mutation functionally inactivates, or renders non-functional and/or inoperative, a naturally occurring nucleic acid sequence for expression.

As used herein, a “knockout” or “KO” is used to refer to genetic manipulation, wherein the manipulation results in a gene being made nugatory and/or the function of the gene is eliminated, either mostly or completely. A knockout may be achieved through various methods known in the art, for example, integration of a premature stop codon or insertions and/or deletions to the degree of rendering the gene inoperative.

As used herein, a “knockdown” or “KD” is used to refer to genetic manipulation, wherein the manipulation results in a gene's expression being reduced. A knockdown may be achieved through use of genetic modification resulting in the reduced transcription of a gene or by use of introducing an exogenous polypeptide encoding a short DNA or RNA oligonucleotide(s) that have a sequence complementary to either the gene or an mRNA transcript resulting in lack of abundance of functional gene transcript.

The terms “exogenous” and “heterologous” are used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

In an aspect, the disclosure provides a purified cell composition comprising one or more of the transgenic cells of the disclosure.

### Methods for Genetic Editing and Related Matters

Genome editing tools may be used to engineer and/or manipulate cells. In some embodiments, the immune cell of the disclosure may be engineered with either CRISPR, TALEN, or ZFN genome editing tools.

Genome editing tools such as the clustered regularly interspaced short palindromic repeats (CRISPR) system may be used to genetically modify cells. CRISPR can be used in a wide variety of organisms (e.g., used to add, disrupt, or change the sequence of specific genes). “CRISPR” or “CRISPR gene editing” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence, knock out, or mutate a target gene.

The CRISPR/Cas system is based on two elements. The first element is an endonuclease, or Cas, (e.g., Cas9 and MAD7) that has a binding site for the second element, which is the guide polynucleotide (e.g., guide RNA or gRNA). The guide polynucleotide (e.g., guide RNA) directs the Cas protein to double stranded DNA templates based on sequence homology. The Cas protein then cleaves that DNA template. By delivering the Cas protein and appropriate guide polynucleotides (e.g., guide RNAs) into a cell, the organism's genome is cut at a desired location. Following cleavage of a targeted genomic sequence by a Cas/gRNA complex, one of two alternative DNA repair mechanisms can restore chromosomal integrity:

1) non-homologous end joining (NHEJ) which generates insertions and/or deletions of a few base-pairs (bp) of DNA at the gRNA cut site, or 2) homology-directed repair (HDR) which can correct the lesion via an additional “bridging” DNA template that spans the gRNA cut site. CRISPR/Cas systems are classified by class and by type. Class 2 systems currently represent a single interference protein that is categorized into three distinct types (types II, V and VI). Any class 2 CRISPR/Cas system suitable for gene editing, for example a type II, a type V or a type VI system, is envisaged as within the scope of the instant disclosure. Exemplary Class 2 type II CRISPR systems include Cas9, Csn2 and Cas4. Exemplary Class 2, type V CRISPR systems include, Cas12, Cas12a (Cpf1), Cas12b (C2c1), Cas12c (C2c3), Cas12d (CasY), Cas12e (CasX), Cas12f, Cas12g, Cas12 h, Cas12i and Cas12k (C2c5). Exemplary Class 2 Type VI systems include Cas13, Cas13a (C2c2) Cas13b, Cas13c and Cas13d.

The CRISPR sequence, sometimes called a CRISPR locus, comprises alternating repeats and spacers. In a naturally-occurring CRISPR, the spacers usually comprise sequences foreign to the bacterium such as a plasmid or phage sequence. As described herein, spacer sequences may also be referred to as “targeting sequences.” In CRISPR/Cas systems for a genetic engineering, the spacers are derived from the target gene sequence (the gNA).

The targeting sequence can be designed or chosen using computer programs known to persons of ordinary skill in the art. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like. Available computer programs can take as input NCBI gene IDs, official gene symbols, Ensembl Gene IDs, genomic coordinates, or DNA sequences, and create an output file containing sgRNAs targeting the appropriate genomic regions designated as input. The computer program may also provide a summary of statistics and scores indicating on- and off-target binding of the sgRNA for the target gene (Doench et al. Nat Biotechnol. 34:184-191 (2016)).

The target sequence is complementary to, and hybridizes with, the targeting sequence of the gRNA. The target nucleic acid sequence can comprise 20 nucleotides. The target nucleic acid can comprise less than 20 nucleotides. The target nucleic acid can comprise more than 20 nucleotides. The target nucleic acid can comprise at least: 5, 10, 15, 16, 17, 18, 19, 20, 21, 22, 23, 24, 25, 30 or more nucleotides. The target nucleic acid can comprise at most: 5, 10, 15, 16, 17, 18, 19, 20, 21, 22, 23, 24, 25, 30 or more nucleotides.

The CRISPR/Cas system can thus be used to edit a target gene, such as a gene targeted for editing in the cells described herein, by adding or deleting a base pair, introducing a premature stop codon, or introducing a frame-shift mutation which thus decreases expression of the target, in part or completely. The CRISPR/Cas system can alternatively be used like RNA interference, turning off a target gene in a reversible fashion. In a mammalian cell, for example, the RNA can guide the Cas protein to a target gene promoter, sterically blocking RNA polymerases.

Further aspects of the CRISPR/Cas system known to those of ordinary skill are described in PCT Publication Nos. WO 2017/049266 and WO 2017/223538, the entire contents of which are hereby incorporated by reference. These and other well-known and new techniques, such as TALEN and Zinc Finger Nucleases, for generating mutant cells of the present disclosure are contemplated by the present invention.

In some embodiments, the mutant cells described herein are edited using TALEN gene editing. “TALEN” or “TALEN gene editing” refers to a transcription activator-like effector nuclease, which is an artificial nuclease used to edit a target gene. TALENs are produced artificially by fusing a TAL effector DNA binding domain to a DNA cleavage domain. Transcription activator-like effectors (TALEs) can be engineered to bind any desired DNA sequence, including a portion of target genes such as TCR subunits, MHC class I complex components, or CD52. By combining an engineered TALE with a DNA cleavage domain, a restriction enzyme can be produced which is specific to any desired DNA sequence, including a target gene sequence. These can then be introduced into a cell, wherein they can be used for genome editing. Boch (2011) Nature Biotech. 29:135-6; and Boch et al. (2009) Science 326: 1509-12; Moscou et al. (2009) Science 326:3501.

In some embodiments, the mutant cells described herein are edited using ZFN gene editing. “ZFN” or “Zinc Finger Nuclease” or “ZEN gene editing” refer to a zinc finger nuclease, an artificial nuclease which can be used to edit a target gene. Like a TALEN, a ZFN comprises a Fold nuclease domain (or derivative thereof) fused to a DNA-binding domain. In the case of a ZFN, the DNA-binding domain comprises one or more zinc fingers. Carroll et al.

(2011) Genetics Society of America 188:773-782; and Kim et al. (1996) Proc. Natl. Acad. Sci. USA 93:1156-1160.

The term “nucleic acid” or “polynucleotide” includes DNA and RNA such as genomic DNA, cDNA and mRNA, or combinations thereof. The nucleic acid may comprise, in addition to the sequence enabling the genetic modifications of the disclosure, further sequences such as those required for the transcription and/or translation of the nucleic acid enabling said genetic modifications. This may include a promoter, enhancer, transcription and/or translation initiation and/or termination sequences, selection markers, sequences protecting or directing the RNA and/or enabling the genetic modifications within the cell. The selection and combination of these sequences is within the knowledge of the person skilled in the art and may be selected in accordance with the cell the nucleic acid is intended for.

Polynucleotides enabling the genetic modifications of the disclosure may be delivered to cells as an isolated nucleic acid or in a vector. The isolated nucleic acid or the vector may be delivered in lipid- or lipid-based delivery system, such as a liposome. Alternatively, the vector may comprise viral proteins, such as when the vector is a viral vector. The term “vector” as used herein refers to a construction comprised of genetic material designed to direct transformation or transductions of a targeted cell. A vector contains multiple genetic elements positionally and sequentially oriented with other necessary elements such that the nucleic acid in a nucleic acid cassette can be transcribed and when necessary translated in the transfected cells. The term vector as used herein can refer to nucleic acid, e.g., DNA derived from a plasmid, cosmid, phagemid, bacteriophage, virus, retrovirus, adenovirus, adeno-associated virus, lentivirus, or other type of virus into which one or more fragments of nucleic acid may be inserted or cloned which encode for particular proteins. The term “plasmid” as used herein refers to a construction comprised of extrachromosomal genetic material, usually of a circular duplex of DNA which can replicate independently of chromosomal DNA. The plasmid does not necessarily replicate.

Any suitable vectors are envisaged as within the scope of the instant disclosure. The polynucleotides enabling the genetic modifications of the disclosure can be cloned into a number of types of vectors. For example, the polynucleotides enabling the genetic modifications of the disclosure may be cloned into a vector including, but not limited to a plasmid, a phagemid, a phage derivative, an animal virus, and a cosmid. Vectors of particular interest include expression vectors, replication vectors, probe generation vectors, and sequencing vectors. Expression vectors may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

The purpose of the vector is to provide a nucleic acid sequence in cells or tissue. Expression includes the efficient transcription of an inserted gene or nucleic acid sequence. Expression products may be proteins, polypeptides, or RNA. The nucleic acid sequence can be contained in a nucleic acid cassette. Expression of the nucleic acid can be continuous, constitutive, or regulated. The vector can also be used as a prokaryotic element for replication of plasmid in bacteria and selection for maintenance of plasmid in bacteria.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, transducing comprises either calcium phosphate-mediated gene transfer, DEAE-dextran-mediated gene transfer, liposome-mediated gene transfer, electroporation-mediated gene transfer, viral vector-mediated gene transfer, or nucleofection-mediated gene transfer. In some embodiments, transducing is accomplished by calcium phosphate-mediated gene transfer. In some embodiments, transducing is accomplished by liposome-mediated gene transfer. In some embodiments, transducing is accomplished by electroporation-mediated gene transfer. In some embodiments, transducing is accomplished by viral vector-mediated gene transfer. In some embodiments, transducing is accomplished by nucleofection-mediated gene transfer.

Regardless of the method used to introduce exogenous nucleic acids into a host cell, in order to confirm the presence of the recombinant DNA sequence in the host cell, or confirm effect of genomic modulation, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays.

The invention may also refer to any oligonucleotides (antisense oligonucleotide agents), polynucleotides (e.g. therapeutic DNA), ribozymes, DNA aptamers, dsRNAs, siRNA, RNAi, and/or gene therapy vectors. The term “antisense oligonucleotide agent” refers to short synthetic segments of DNA or RNA, usually referred to as oligonucleotides, which are designed to be complementary to a sequence of a specific mRNA to inhibit the translation of the targeted mRNA by binding to a unique sequence segment on the mRNA. Antisense oligonucleotides are often developed and used in the antisense technology. The term “antisense technology” refers to a drug-discovery and development technique that involves design and use of synthetic oligonucleotides complementary to a target mRNA to inhibit production of specific disease-causing proteins. Antisense technology permits design of drugs, called antisense oligonucleotides, which intervene at the genetic level and inhibit the production of disease-associated proteins. Antisense oligonucleotide agents are developed based on genetic information.

As an alternative to antisense oligonucleotide agents, ribozymes or double stranded RNA (dsRNA), RNA interference (RNAi), and/or small interfering RNA (siRNA), can also be used as therapeutic agents for regulation of gene expression in cells. As used herein, the term “ribozyme” refers to a catalytic RNA-based enzyme with ribonuclease activity that is capable of cleaving a single-stranded nucleic acid, such as an mRNA, to which it has a complementary region. Ribozymes can be used to catalytically cleave target mRNA transcripts to thereby inhibit translation of target mRNA. The term “dsRNA,” as used herein, refers to RNA hybrids comprising two strands of RNA. The dsRNAs can be linear or circular in structure. The dsRNA may comprise ribonucleotides, ribonucleotide analogs, such as 2′-O-methyl ribosyl residues, or combinations thereof. The term “RNAi” refers to RNA interference or post-transcriptional gene silencing (PTGS). The term “siRNA” refers to small dsRNA molecules (e.g., 21-23 nucleotides) that are the mediators of the RNAi effects. RNAi is induced by the introduction of long dsRNA (up to 1-2 kb) produced by in vitro transcription, and has been successfully used to reduce gene expression in variety of organisms. In mammalian cells, RNAi uses siRNA (e.g. 22 nucleotides long) to bind to the RNA-induced silencing complex (RISC), which then binds to any matching mRNA sequence to degrade target mRNA, thus, silences the gene.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the art.

“Codon” refers to a sequence of three nucleotides that together form a unit of genetic code in a nucleic acid.

“Complementary” or “complement thereof” means that a contiguous nucleic acid base sequence is capable of hybridizing to another base sequence by standard base pairing (hydrogen bonding) between a series of complementary bases. Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997)” “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

### EXAMPLES

### Results

### Establishing Transgenic CRISPR Lines to Target Femaleless

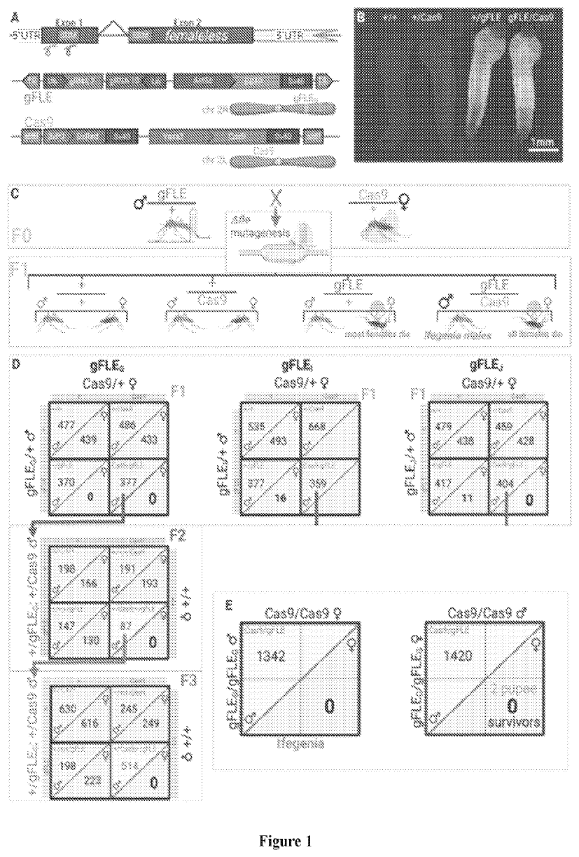

It was hypothesized that embryonic biallelic knockout of the femaleless (fle) gene could cause female death, so a binary CRISPR-based technology to target this gene for embryonic mutagenesis was developed. A gRNA-expressing transgene to express two gRNA's targeting the N-terminal region of fle was designed and cloned. One gRNA targeted 25 bp downstream of the start codon (“gRNA 7”), and the second targeted the first RNA Recognition motif (RRM) (“gRNA 10”) (FIG. 1, Panel A). Three distinct families (termed gFLEG, gFLE_{I}, gFLE_{J}) were established by piggyBac transgenesis and chromosomal integration was confirmed by the presence of an Act5C-GFP selectable marker. It was hypothesized that early embryonic and larval targeting of fle would yield strong killing effects. Therefore, for Cas9 expression, the transgenic line, Vasa2-Cas9, previously described for its robust germline mutagenesis (FIG. 1, Panel A) 34, henceforth shortened to Cas9 hereon for brevity, was selected.

### Elimination of Daughters

To determine the phenotype of gFLE/Cas9 trans-heterozygotes genetic crosses between gRNA-positive males to Cas9 homozygous females were performed and F1 offspring were analyzed. Strikingly, it was observed that all F1 transheterozygous gFLE/Cas9 pupae identified in preliminary experiments, regardless of family, were phenotypic males (N=638, Table 1) suggesting females were killed or androgenized before pupation. To confirm mutations in femaleless may be the cause of this phenotype, gFLEG/Cas9 adults were sequenced and identified multiple mutations under both gRNA target sites (data not shown). More mutations were identified at the gRNA 10 site than the gRNA 7 site. Quantitative RT-PCR confirmed transcript elimination of fle in gFLE/Cas9 adults, suggesting knockout of femaleless eliminates females prior to pupation.

TABLE 1
No transheterozygous gFLE/Cas9 females
were observed from preliminary crosses.

| | Males | Females |
|---|---|---|
| Genotype | Observed (n) | Observed (n) |
| gFLE_{G}/Cas9 | 292 | 0 |
| gFLE_{I}/CAS9 | 179 | 0 |
| gFLE_{J}/Cas9 | 167 | 0 |

### Fle is Essential for Females Early in Development

To determine if fle knockout was killing females, or androgenizing them to be indistinguishable from males, heterozygous gRNA/+ males to heterozygous Cas9/+ females were crossed and the sexes and genotypes of all resulting F1 offspring were quantified (FIG. 1, Panels B and C). Androgenization would be expected to be observed as an increase in males in mutating genotypes versus non-mutating control siblings, while female killing would be observed as the absence of females from otherwise equal groups. The resulting Punnett Squares demonstrated that F1 gFLE/Cas9 males were present at roughly equal ratios to control siblings (whereas androgenization would have been observed as an increase (e.g., doubling) of gFLE/Cas9 individual compared to other groups (FIG. 1, Panel D). Additionally, no F1 gFLE/Cas9 adult genetic females were observed (here or in any family during the course of all experiments, nor in subsequent F2 and F3 generations). These results suggested that gFLE/Cas9 females were being killed rather than androgenized (FIG. 1, Panel C). To further support this, PCR was performed on gFLE/Cas9 adults and confirmed the presence of a Y chromosome in all individuals assayed, suggesting all individuals were genetically male. Interestingly, heterozygous gFLE/+ females were also completely absent from family gFLEG, and gFLE_{I}/+ and gFLE_{J}/+ showed an approximately 23- and 38-fold reduction in females respectively relative to brothers of that genotype (n=377:16, n=417:11) (FIG. 1, Panel D). This suggests that maternally deposited Cas9, either as RNA or protein, alone is sufficient for fle mutagenesis and female killing when only the gFLE transgene is inherited. Interestingly, slightly fewer females than males were present among+/+ and +/Cas9 control siblings, possibly suggesting low levels of embryo mutagenesis enabled by paternal deposition of gRNAs (FIG. 1, Panel D) 25.

During the course of these experiments some female pupae developed in the +/gFLE_{I} and +/gFLE_{J} genotype (FIG. 1, Panel D), suggesting attenuated transgene penetrance and warranting exclusion of these two families from further analysis. Strikingly however, no gFLE/Cas9 adult females were observed in any family during the course of all experiments, despite 2,512 total gFLE/Cas9 males scored. This demonstrates that CRISPR targeting of femaleless is exceedingly robust displaying extreme penetrance of female killing, and proves that this technology should be considered a GSS candidate for anopheline vector control campaigns. This system is termed Ifegenia for (Inherited Female Elimination by Genetically Encoded Nucleases to Interrupt Alleles) herein to simplify discussion and reference of the technology.

Because gFLEG yielded the strongest F1 hybrid female-killing phenotype among gFLE families, this line was proceeded for further characterization. For this, transgene insertion sites and fle mutation profiles of gFLEG/Cas9 males were characterized by nanopore DNA sequencing, confirming a single transgene insertion and expected Δfle alleles. To determine relative fle expression, RNAseq on 3 replicates of 18 hr old embryos enriched for the gFLEG/Cas9 genotype was performed compared to 3 replicates of each control genotype gFLEG, Cas9 and WT. A modest but highly significant reduction in fle was observed (padj ranging from 4E-06-2E-08) with non-mutant genotypes clustering together as expected, with no observable change in dsx.

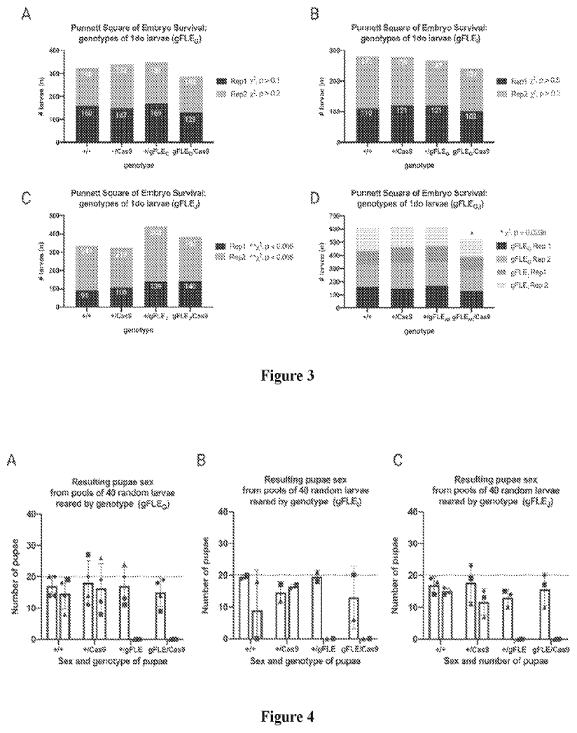

Next, to determine if female death was occurring during embryogenesis, the one day old larval genotypes of F1 larvae resulting from the cross outlined in FIG. 1, Panel C were quantified. Only a very slight reduction in the quantity of larvae present in the gFLE/Cas9 genotype (FIG. 3, Panel D) was observed, suggesting the majority of the female cohort of gFLE/Cas9 survived embriogenesis. To determine if females were killed as larvae, F1 larvae from hatching to pupation were monitored. While the majority +/+ and +/Cas9 siblings survived larva hood, only half of F1+/gFLE and gFLE/Cas9 individuals survived, all of which were male, suggesting significant female death during larval life-stages (FIG. 4). These findings are consistent with aberrant X chromosome dosage compensation phenotypes observed in other Dipterans, which was confirmed by RNAseq.

### Ifegenia Males are Fit, Reproductively Viable, and Pass on ΔFle Alleles

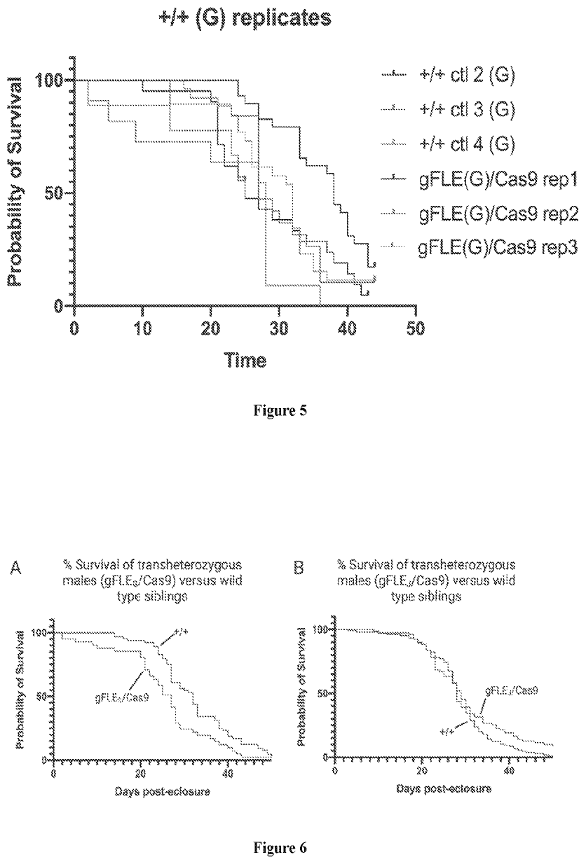

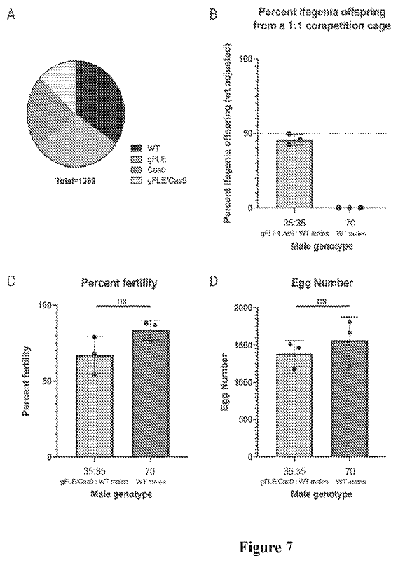

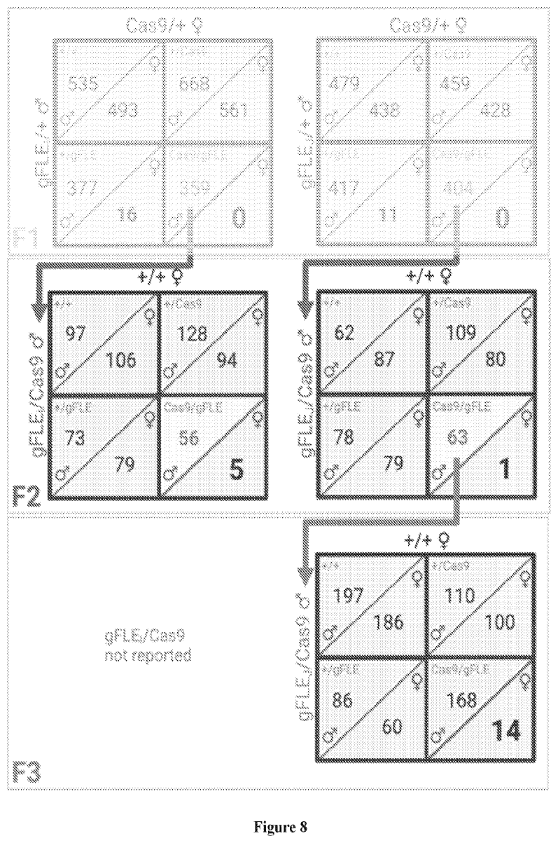

For population suppression, Ifegenia requires that released adult males be fit and reproductively viable in order to mate, and pass on both heritable mutant alleles and CRISPR components to subsequent generations. Adult male fitness was confirmed by survival curves (FIG. 5 (individual replicates) and FIG. 6 (pooled data)). To quantify adult longevity, gFLEG/Cas9 and gFLE_{J}/Cas9 males were monitored and compared to +/+ siblings in survival assays. Male gFLEG/Cas9 (FIG. 6, Panel A), but not gFLE_{J}/Cas9 (FIG. 6, Panel B), displayed slight reductions in longevity. To verify reproductive viability, Ifegenia males were crossed to wild type females and confirmed the presence of viable progeny. Mating competition assays were then performed between equal numbers of Ifegenia and WT males, expecting 50% Ifegenia offspring if males were equally competitive. Nearly equal percent Ifegenia progeny as WT progeny (45.7%) were observed indicating robust mating competitiveness (FIG. 7). Together these results suggest that Ifegenia males should be sufficiently reproductively viable to achieve population suppression following iterative releases, however large-scale cage and field trials should be undertaken in the future.

### Ifegenia Induces Genetic Sexing

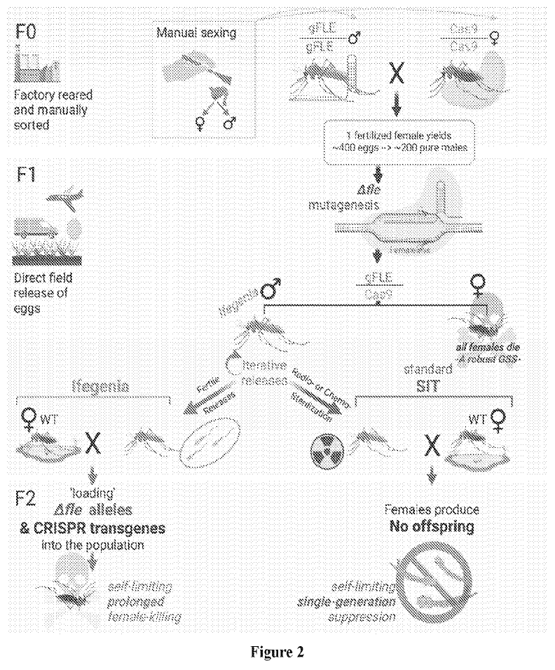

Given the profound female-killing properties of Ifegenia, its penetrance of genetic sexing for use in male-only vector control campaigns (FIG. 2) was desired to be determined. Homozygous gFLEG/gFLEG males were mated to Cas9/Cas9 females, and vice versa, and verified the complete absence of females among all offspring (FIG. 1, Panel E). As expected, complete killing of genetic females was observed before pupal eclosure (N=1342 gFLEG/Cas9 males from maternal Cas9 and N=1420 males from paternal Cas9). Interestingly however, a single, likely sterile, phenotypic gFLEG/Cas9 female as defined by the pupal genitalia parameters of our assay was identified. This individual PCR-amplified for the Y-chromosome revealing a rare male-feminization intersex genotype. Taken together, no evidence of viable F1 females in the gFLEG/Cas9 lines used in this work was observed, revealing a powerful application of Ifegenia as a tool for genetic sexing of A. gambiae (FIG. 2).

Ifegenia males confer multi-generational daughter gynecide

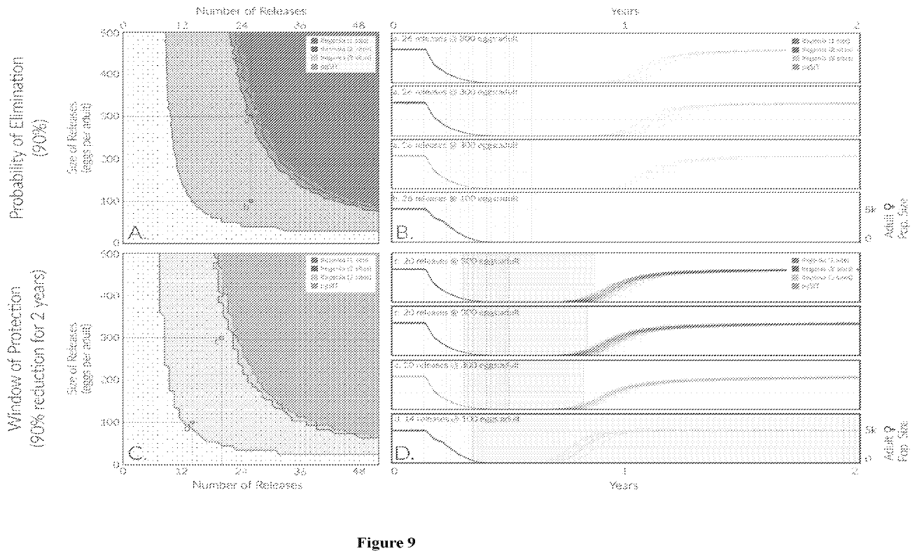

It was next determined if Ifegenia males can ‘load’ non-driving CRISPR transgenes and Δfle alleles into subsequent generations (F2 and F3), and cause multi-generational daughter-killing effects (FIG. 1, Panel D and FIG. 9). Transmission of transgenes at ratios consistent with linkage of the gFLE and Cas9 transgenes on the 2nd chromosome, and complete female killing of all F2 and F3 gFLEG/Cas9 females (FIG. 1, Panel D) was observed. Moreover, out of the few F2 and F3 gFLE/Cas9 females observed from families gFLE_{I} and gFLE_{J}, all either died as pupae or young adults, appeared androgenized, or were feminized XY genetic males. The approximate frequency of Δfle alleles in the F2 generation by PCR and enzyme digest³⁶ was also determined. An 85-95% Δfle allele frequency among F2 individuals inheriting one or no transgenes was observed, representing a conservative estimate on the frequency of germ cell mutagenesis in the parental F1 gFLEG/Cas9 germline. Importantly, a number of individuals were sequenced for the mutant allele, providing evidence of heterozygous mutagenesis in fle (Δfle), indicating that fle is likely haplosufficient, in contrast to earlier hypotheses that it was haplolethal³³. Among F2 progeny of the gFLEG/Cas9 genotype, 100% harbored at least one Δfle allele, however this is likely inflated due to active mosaic mutagenesis occurring in this genotype^{37,20}. Together these results indicate that Ifegenia males are reproductively viable, and pass on both CRISPR transgenes and Δfle alleles to subsequent generations resulting in multi-generational daughter gynecide.

### Ifegenia Induces Confinable Population Suppression

To determine whether iterative releases of fertile Ifegenia males could facilitate population suppression and elimination, the above data on Ifegenia performance was incorporated into a mathematical model³⁸ and simulated releases into a population of 10,000 A. gambiae adults (FIG. 9) with life-history parameters described in Table 2. Weekly releases of up to 500 Ifegenia eggs (female and male) per wild-type adult (female and male) were simulated over 1-52 weeks. The scale of these releases was chosen considering adult release ratios of 10:1 are common for sterile male mosquito interventions³⁹ and female A. gambiae produce >30 eggs per day in temperate climates⁴⁰. Ifegenia constructs with 1-3 gRNA target sites were considered and, to be conservative, simulated target site cutting rates of 90% per allele, maternal deposition of Cas9 in 90% of embryos when expressed by the mother, and male mating competitiveness of 75% that of wild-type males. Results from these simulations suggest that substantial population suppression (≥90%) that endures for ≥2 years is observed for a wide range of achievable release schemes, e.g., >22 weekly releases of 300 or more Ifegenia eggs per wild adult, and elimination is expected to occur in ≥90% of simulations for >26 weekly releases of the same size.

TABLE 2
Parameters used in Anopheles gambiae population suppression model.

| | Parameter: | Value (range): | Reference: |
|---|---|---|---|
| Ifegenia & | gRNA cleavage frequency per allele | 0.9 | |
| pgSIT: | Number of gRNA target sites | 1-3 | |
| | Maternal deposition of Cas into embryo | 0.9 | |
| | Viability of females homozygous for | 0 | |
| | any fle-ko mutant allele | | |
| | Fertillity of males homozygous for | 0 | Li et al.³⁷ |
| | male fertility mutant allele | | |
| | Lifespan reduction of transgenic females & males | 0 | |
| | Male mating competitiveness of transgenic males | 0.75 | |
| | Number of consecutive weekly releases | 26 (1-52) | Kandul et al.²⁰ |
| | Number of pgSIT eggs released per wild adult | 300 (0-500) | Kandul et al.²⁰ |
| Mosquito | Egg production per female (per day) | 32 | Depinay et al.⁴⁰ |
| life history: | Duration of egg stage (days) | 1 | Depinay et al.⁴⁰ |
| | Duration of larval stage (days) | 13 | Depinay et al.⁴⁰ |
| | Duration of pupal stage (days) | 1 | Depinay et al.⁴⁰ |
| | Daily population growth rate (per day) | 1.096 | Molineaux & |
| | | | Gramiccia⁶⁰ |
| | Daily mortality risk of adult stage (per day) | 0.15 | Molineaux & |
| | | | Gramiccia⁶⁰ |
| Landscape | Number of adult mosquitoes | 10,000 | Taylor et al.⁶¹ |

Ifegenia performance is robust to several system features—a sensitivity analysis revealed that the “window of protection” model outcome (the duration for which the population is suppressed by ≥90%) depends very little on the cutting rate, rate of maternal deposition of Cas, and mating competitiveness of males having the construct. FIG. 9 reveals that the number of target sites also has little impact on modeled suppression outcomes; however, a supplementary analysis in which resistant allele generation is considered revealed how increasing the number of target sites greatly reduces the rate at which resistant alleles propagate in the population. These findings suggest that Ifegenia can achieve robust temporary population suppression over a wide range of release parameter values, permitting rebound of native populations after ceasing releases. Opportunity for population rebound may be desired in locales where disease elimination has been achieved, and where ecological concerns warrant return of the native mosquito, making Ifegenia a valuable vector control technique for the toolkit.

### DISCUSSION

The present disclosure characterizes mosaic CRISPR knockout of fle in A. gambiae and demonstrate its potential as a genetic sex sorting system and novel suppression technology. The instant findings illustrate that using a bicomponent CRISPR system to target fle results in the complete killing of genetic females before eclosure through lethal mosaicism^{20,37} and leaves F1 male siblings fit to mate and sire offspring^{41,42}. Termed Ifegenia, its strong female-killing phenotype makes it suitable, for example, for vector control campaigns that require male-only releases. Thus, two exemplary implementations for Ifegenia are suggested: 1) as a genetic sexing system (GSS) to be combined with other vector control methods, and 2) as an independent population suppression vector control technology

Historically, creating a scalable GSS in anophelines has been challenging, with many systems displaying low penetrance^{23,28} or requiring manual or fluorescent-based sex-sorting directly prior to release^{18,27}. Developing inducible genetic systems that automatically eliminate the undesired sex have been difficult due to limited understanding of the sex-determination pathway⁴³, and husbandry limitations have prohibited development of translocation lines²⁹. Overcoming these hurdles, Ifegenia's design offers 4 key advantages that enable scalable production. One, the two homozygous F0 stock strains have normal sex ratios and fertility, enabling mass rearing. Two, a single F0 fertilized female yields conservatively 200 Ifegenia sons, facilitating large-scale production. Three, the separation of gFLE and Cas9 stock lines prevents the creation of CRISPR-resistant alleles, circumventing roadblocks faced by some gene drives⁴⁴. And four, the high phenotype penetrance enables direct release of F1 eggs/larvae/pupae into the wild, eliminating the need for injurious manual sorting and resources for rearing females. Where Ifegenia lacks scalability is the requirement for manual sorting to establish the F0 parental cross. A hurdle easily overcome by the addition of sex-specific fluorophores^{13,14,16-18}, making Ifegenia's throughput sufficient for most applications.

As an efficient and potentially scalable GSS, Ifegenia could help enable technologies like SIT in A. gambiae⁴⁵. Traditional SIT entails committing to mass iterative releases of sterile males to suppress the local populations, but has not yet been adapted to A. gambiae. After years of optimization, radio-sterilization protocols that once reduced male fitness are now ready for larger trials, thus necessitating a male-only GSS in the species^{8,46}, and presenting a niche that could be fulfilled by Ifegenia.

In addition to serving as a GSS, Ifegenia could be used as a novel vector control technology. The modeling described herein suggests that iterative releases of Ifegenia males leads to long-term suppression in wild populations without drive following the introduction of CRISPR transgenes and female-killing alleles. Ifegenia is thus proposed as, for example, an intermediate between control methods like (pgSIT)³⁷ and suppression gene drives^{30,47}. Unlike pgSIT, Ifegenia releases transgenes into the gene pool and induces multigenerational suppression, but unlike gene drives, it is not self-propagating. This could make Ifegenia a desirable option in remote areas where repeated SIT releases are not feasible and gene drives have yet to gain acceptance. While resistant alleles could develop, this risk is reduced by targeting two different loci in fle. If resistant alleles persist, a system with multiplexed targeting of additional female-essential genes could be developed, which modeling suggests should achieve similar performance as targeting fle alone.

In this context, Ifegenia is comparable to female-specific flightless RIDL (fsRIDL), which similarly aims for male-only releases, induces female-killing, and has transgenes that persist for prolonged suppression. fsRIDL involves males passing down a repressible, toxic, female-specific flight muscle transgene to their daughters, preventing them from flying. While fsRIDL has been successful in suppressing Aedes populations in trials⁴⁸ and is slated to be used in the US⁴⁹, this design has not yet been reproduced in A. gambiae. Despite some differences in larval competition parameters and transgene number, its similarities with fsRIDL suggest that Ifegenia could achieve parallel outcomes in the field.

Assuming the commitment for iterative releases has been made, releases of fertile Ifegenia males could act as a novel population suppression technology (e.g., by siring Δfle offspring). In such a system, iterative mass release of gFLE/Cas9 Ifegenia males could still cause a reduction in the biting population in subsequent generations much like traditional SIT, however it would additionally ‘load’ the population with female-killing Δfle alleles and fle-targeting CRISPR machinery-theoretically permitting for prolonged population suppression effects. Because these releases are entirely non-driving, but still entail introduction and transient persistence of GM mosquitoes in the environment, they could be examined as an intermediate vector control option between the alternative suppression technologies pgSIT and Suppression Gene Drives.

A. gambiae is the predominant vector for malaria. Vector population suppression technologies offer a promising means to curb malaria transmission. However, traditional technologies like insecticides rapidly grow ineffective because Anopheline mosquitoes, which have high sequence variation due to a high mutation rate, quickly develop insecticide resistance. Technologies that involve releasing sterile males, such as pgSIT and fsRIDL, have been previously demonstrated in D. melanogaster and the malaria vector Aedes aegypti to confineably suppress population for a single generation. These genetic systems involve releasing sterile males into the wild to compete against wild type males for mates. The cross between a sterile male and a wild type female fails to produce viable progeny, resulting in single-generation, confineable population suppression. One cornerstone for this genetic system is that the males are fit and mate with wt females to deliver a mating plug that prevents further matings. Also important, fertile females should not be released alongside the sterile males, because females can blood feed and contribute to disease transmission. Thus, these genetic systems knock out some genes essential for male fertility and other female-specific genes essential for female development, in order to solely generate sterile males for release.

Limited characterization of the A. gambiae sex determination pathway makes it difficult to find female-specific gene targets. A previous study recently published a RNAi knockdown characterization of the novel gene, fle, in A. gambiae, which suggests female-specific lethality and androgenization. Here, in the first study to characterize fle knockout, the utility of fle as a gene target in pgSIT-like systems is demonstrated. A pgSIT-like crossing scheme to knock out fle was used, and demonstrated that fle knockout results in complete female elimination without apparently affecting the ability of their male siblings to mate. In fact, the loss of female phenotype resulting from fle knockout is more penetrant than the previously only known gene target, Dsx, which has varying levels of androgenization and leakiness. Based on these results, fle is a promising gene target to incorporate into sterile male technologies.

This disclosure highlights the potential for fle-targeting technologies to enable new genetically modified vector control methods. High genetic conservation could permit trivial adaptation of Ifegenia and other fle-based tools to the related species A. arabiensis, A. coluzzii, and A. stephensi³³ to curb their impact on malaria transmission. Ifegenia could facilitate further study of fle, yielding insights into its role in X-chromosome dosage compensation, and the biological function and of its homolog Transformer 2⁵⁰, potentially enabling novel vector control systems in harmful agricultural pests such as C. capitata, and L. cuprina⁵¹. Finally, manipulating fle reveals opportunities for tet-based sex separation systems, homing based gene drives⁵²⁻⁵⁴, and Y-drives³¹ in addition to other tools, making possible a suite of new vector control technologies to target a range of pests. In all, Ifegenia not only provides important insights on the function of fle, and demonstrates its value as a gene of interest for the vector control field at large, but also provides a GSS, and the first tool of its kind to combat malaria transmission in Anopheles gambiae.

### Materials and Methods

### Mosquito Rearing and Maintenance

A. gambiae was derived from the G3strain. The mosquitoes were reared in 12 h light/dark cycles, in cages (Bugdorm, 24.5×24.5× 24.5 cm). The cages were placed in a transparent plasmid bag to maintain humidity and allow light to go through. For every 2 cages, 1 humidity cup was placed on top of the cage to maintain high humidity in the bag. Adults were provided with 0.3 M aqueous sucrose ad libitum, and females were blood fed on anesthetized mice for 2 consecutive days for ˜15 min at a time. Males and females were allowed to mate for at least 2 days before blood meal. Egg dishes are provided 2 days post-blood meal. Eggs are allowed to melanize for 2 days before being floated in trays. Larvae were reared and fed, as well as pupae screened and sexed, in accordance with established protocols⁵⁰.

The fle (AGAP013051) target gene reference sequence was extracted from VectorBase⁴⁸. To verify target sequence and detect any polymorphisms, gDNA was extracted (Qiagen, DNeasy Blood & Tissue Kits, Cat. No./ID: 69504) from pools of 10 individuals, cloned into pJET (ThermoFisher Scientific, Cat. No./ID: K1231), and individual colonies were sanger sequenced for the fle locus. Putative candidate gRNAs in conserved regions were identified using hcrispor.tefor.net/. On-target in vitro cleavage activity was determined for the top ten gRNA candidates by CRISPR QC. The gRNAs tested are as follows gRNA1 [5′ TCATCCGCTTTCGACACTCGCGG 3′] (SEQ ID NO: 1); gRNA2 [5′ CACGATGAGTATTGAGTCTTGG 3′] (SEQ ID NO: 2); gRNA3 [5′ TGCTTCGTACAGCTGCCAGTCGG 3′] (SEQ ID NO: 3); gRNA4 [5′ CTTCCACCGGCGGTAATCTTTGG 3′] (SEQ ID NO: 4); gRNA5 [5′ TTATCACATTGTATGACGGTCGG 3′] (SEQ ID NO: 5); gRNA6 [5′ GTGCTGGACGCATTCCTATTGGG 3′] (SEQ ID NO: 6); gRNA7 [5′ CGACGGCTCGTTCATCGCTGGGG 3′] (SEQ ID NO: 7); gRNA8 [5′ GTCGACGGCTCGTTCATCGCTGG 3′] (SEQ ID NO: 8); gRNA9 [5′ CTTGAACAGCTCTATCAGATCGG 3′] (SEQ ID NO: 9); gRNA10 [5′ ATCGAGCGCGTCGCCTGGTACGG 3′] (SEQ ID NO: 10). Two gRNAs, gRNA7 and gRNA10 were selected as each targets Exon 1, and overlaps a semi-unique restriction enzyme site to facilitate downstream screening and identification of mutant alleles⁵⁵. A piggyBac transgenesis backbone pbVTKactR⁵⁶ was modified to contain each gRNA under expression of the A. gambiae U6 promoter³⁴ synthesized as gBlocks®, and replaced endogenous Red for m2Turquoise (ECFP) The final plasmid sequence (plasmid 1154B, transgene gFLE) was confirmed by Sanger sequencing and is available on Addgene (#187238). Embryonic microinjections of gFLE into G3 wild type embryos was carried out as described previously¹⁴. 9 transgenic founders were identified, which were individually outcrossed to wild type to establish distinct families. Families gFLEG, gFLE_{I}, and gFLE_{J} were selected for analysis.

### Fluorescent Sorting, Sexing and Imaging

A. gambiae were fluorescently sorted, sexed, and imaged using the Leica M165FC fluorescent stereomicroscope using a Leica DMC2900 camera. Fluorescence was visualized using the CFP/YFP/mCherry triple filter, and was sexed by examination of pupal genital terminalia. In cases where sex was indeterminable by pupal phenotype, genotype was validated by Y-chromosome PCR (see below).

### Genetic Cross Setup

For all crosses, pupae were fluorescently sorted and sexed, and allowed to emerge as adults in separate cages to ensure female virginity before crossing. Unless otherwise indicated, crosses were set up with 1-3 day old adults, allowed to mate ad libitum for 4 days, then blood fed. For preliminary test crosses, 50 gFLE-positive males and 50-Cas9 positive females (mixed heterozygotes and homozygotes) were crossed, and offspring pupal sex-genotypes were scored. For crosses requiring heterozygous parents, gFLE-positive or Cas9-positive individuals were first crossed to wild type of the reciprocal sex and fluorescently sorted to generate guaranteed heterozygous F0's. These F0+/gFLE males and +/Cas9 females were then intercrossed for analysis of the Mendelian inheritance patterns in the F1 offspring. For assays requiring homozygous X homozygous mating pairs, one of two assays was set up. In early experiments, small cages of gFLE-positive males (enriched for homozygotes by fluorescence intensity) were mated to pure Cas9/Cas9 females, allowed to mate en masse and allowed to oviposit en masse. Only those broods which yielded 100% gFLE/Cas9 offspring were considered from gFLE/gFLE X Cas9/Cas9, and scored. In a second experiment, larger cross cages were set up essentially as described above, however females were isolated into iso-female ovicups prior to oviposition. Individual broods were screened for pure hybrid transheterozygous (gFLE/Cas9) offspring, and only those with this genotype were scored.

### Embryo Survival Assays

To determine if females were dying during embryogenesis, +/gFLE males and +/Cas9 were crossed. From large egg lays, a random sampling of ˜500 unhatched embryos were separated from the egg dish and allowed to hatch. All larvae were scored by genotype at 1 day post-hatching and reported in FIG. 3.

### Larvae Survival

To determine if females were dying as larvae (after hatching and before pupation), ldo larval offspring of the +/gFLE x+/Cas9 cross were fluorescently sorted. 40 larvae from each genotype were reared in separate trays, then scored as pupae by sex and genotype.

### Fle Knockout Mutant Analysis

DNA was individually extracted from L3-L4 A. gambiae larvae using the DNeasy Blood & Tissue Kits (Qiagen, Cat. No./ID: 69504). 1 μl of genomic DNA was used as a template in a 20 μl PCR reaction using Q5 HotStart DNA polymerase (NEB, Cat. No./ID: M0493 L) and primers 1154A.S5 and 1154A.S7 amplifying genomic fle sequences. The resulting product was run on a 1% agarose gel in TAE buffer), gel extracted with the Zymoclean Gel DNA Recovery Kit (Zymo Research, Cat. No./ID: D4007), cloned into the pJET vector (Thermo Scientific, Cat. No./ID: K1231), transformed into chemically competent E. coli (Promega, JM109), and plated on LB-Ampicillin plates. Sanger sequencing reads from individual colonies represented amplicons from individual mutant alleles, (primers PJET1-2F and/or PJET 1-2R) were compared to fle sequences from our WT mosquitoes as a reference genome.

### Male Mating Competition Assay

Competition cages were set up with 35 gFLE/Cas9 males×35 WT males, or 70 WT males, then introduced into a cage with 35 WT virgin females. They were allowed to mate ad libitum for 5 days before bloodfeeding. Eggs were counted and larvae scored for the presence of a transgene. Because gFLE/Cas9 males sire some non-transgenic progeny as part of normal transgenic chromosome segregation, the ratio of transgenic:WT offspring was calculated (FIG. 7, Panel A) and used to calculate the WT offspring attributable to gFLE/Cas9 fathers in FIG. 7, Panel B.

Δfle allele quantification

To quantify Δfle mutant alleles, PCR amplification was performed followed by direct digest on le amplicons in F2 individuals. Genomic DNA samples were prepared using the Qiagen DNeasy extraction kit (Cat. No./ID: 69504), and amplified in 50 μl PCR reactions using Taq DNA polymerase (NEB, Cat. No./ID: #M0273S) and primers 1154A.S8 and 1154A.S29 (1121 bp). PCR product was divided into three 15 μl aliquots; one was undigested for reference, one digested with BstNI (Cat. No./ID: R0168S), and one with BseYI (NEB cat #R0635S) according to manufacturer's protocols. PCR amplicons from WT alleles are expected to digest into 228 bp and 893 bp, and 403 bp and 718 bp fragments for BseYI (gRNA7) and BstNI (gRNA10) respectively. Failure to digest a significant quantity of PCR product indicates a likely polymorphism under the gRNA target site. Immediately after digestion, the 15 μl raw PCR product, 15 μl BstNI-digested product, and 15 μl BseYI-digested product were run side-by-side on gel (1% agarose gel in TAE buffer, 1 kb ladder (NEB, Cat. No./ID: N3232 L), most gels run at 115V for 40 minutes).

### Y Chromosome Amplification

Samples were extracted with the Qiagen DNeasy extraction kit (Cat. No./ID: 69504). 1 μl of genomic DNA was used as a template in a 20 μl PCR reaction using Q5 HotStart DNA polymerase (NEB, Cat. No./ID: M0493 L). Each individual was genotyped for the presence of the y-chromosome⁵⁷. Positive control PCRs used A. gambiae-specific 28S primers (1123A.S2/1123A.S3, 230 bp fragment) 58.

### Male Survival Curves

Ifegenia males and WT control males were scored as pupae and placed into separate cages. Cages were inspected each day for mosquito death. Dead adults were scored and removed daily, until no surviving adults remained.

### Male Competition Assay

To determine the relative competitiveness of gFLE/Cas9 and wild type males, 35 gFLE/Cas9 males, 35 wild type males and 35 wild type females were crossed together in a single cage in triplicate and assumed monandry. The total number of larvae, number of eggs, and the genotypes of the resulting 1 day old offspring were scored (FIG. 7). To determine the number of offspring from transgenic fathers, crosses of 35 gFLE/Cas9 males to 35 WT females were performed and the ratio of each genotype in the 1 day old offspring was determined (FIG. 7, Panel A). This was used to calculate the approximate number of WT larvae which resulted from gFLE/Cas9 males vs WT males in competition cages. This was reported and graphed as the number of adjusted Ifegenia offspring. Control cages were composed of 70 WT males mated to 35 WT females, and egg number, larvae number, and genotype data were collected in the same manner.

### Mathematical Modeling

To model the expected performance of Ifegenia and pgSIT at suppressing and eliminating local A. gambiae populations, the MGDrivE simulation framework was used⁸. This framework models the egg, larval, pupal, and adult mosquito life stages with overlapping generations, larval mortality increasing with larval density, and a mating structure in which females retain the genetic material of the adult male with whom they mate for the duration of their adult lifespan. The inheritance patterns of the Ifegenia and pgSIT systems were modeled within the inheritance module of MGDrivE. For simplicity, the inheritance model assumes that mosquitoes having the Cas9 allele and a gRNA targeting the N-terminal region of fle, or targeting male fertility in the case of pgSIT, cleave recessive terminal regions of fle and/or of the male fertility allele according to a defined frequency. In the case of multiple target sites, cleavage of each target allele is treated as an independent event. For Ifegenia, females homozygous for any fle mutant allele have reduced viability, and in the case of pgSIT, male mosquitoes homozygous for the fertility mutant allele have reduced fertility. A proportion of progeny of female mosquitoes having the gRNA and Cas9 alleles also have their N-terminal region of fle or, in the case of pgSIT, male fertility allele, cleaved due to the maternal deposition of Cas9. For this analysis, no fitness effects are associated with having the gRNA or Cas9 alleles, except for male mating competitiveness, and individuals heterozygous for either allele are not affected. The development of resistance alleles, which could potentially inhibit the cleavage of gRNA target sites, are accounted for in a supplementary model.

We considered a single randomly-mixing A. gambiae population of 10,000 mosquitoes was considered and the stochastic version of the MGDrivE framework to capture random effects at low population sizes and the potential for population elimination was implemented. Density-independent mortality rates for juvenile life stages were calculated for consistency with the population growth rate in the absence of density-dependent mortality, and density-dependent mortality was applied to the larval stage following Deredec et al.⁵⁹. Weekly releases of up to 500 transgenic eggs per wild adult mosquito (female and male) were simulated over a period of 1-52 weeks. The scale of egg releases was chosen following the precedent in Kandul et al.²⁰ for equivalence to an adult release ratio on the order of 10:1, taking into account survival of released eggs through to the adult life stage in the presence of density-dependent larval mortality. 120 repetitions were carried out for each parameter set, and mosquito genotype trajectories, along with the proportion of simulations that led to local population elimination, were recorded. Complete model and intervention parameters are listed in Table 2.

### Determination of Transgene Integration Sites

To determine the transgene insertion sites, Oxford Nanopore genome DNA sequencing was performed. Genomic DNA was extractedusing the Blood & Cell Culture DNA Midi Kit (Qiagen, Cat. No./ID: 13343) from 16 adult Ifegenia transheterozygous males harboring both transgenes (Cas9/+; gFLE/+), following the manufacturer's protocol. The sequencing library was prepared using the Oxford Nanopore SQK-LSK110 genomic library kit and sequenced on a single MinION flowcell (R9.4.1) for 72 hrs. Basecalling was performed with ONT Guppy base calling software version 6.1.2 using dna_r9.4.1_450bps_sup model generating 3.02 million reads above the quality threshold of Q≥10 with N50 of 12308 bp and total yield of 19.49 Gb. To identify transgene insertion sites, nanopore reads were aligned to plasmids carrying either gFLE (1154B, Addgene as plasmid #187238) or Cas934 constructs using minimap2⁵³. Reads mapped to the plasmids were extracted and mapped to the A. gambiae genome (GCF_000005575.2_AgamP3). Exact insertion sites were determined by examining read alignments in Interactive Genomics Viewer (IGV). The gFLEG transgene is integrated between positions 23,279,556 and 23,279,559 on chromosome 2R (NT_078266.2) and falls into the last intron of AGAP002582. Cas9 is inserted between positions 10,326,500 and 10,326,503 on 2 L (NT_078265.2). The site is located in the intergenic region between AGAP005126 and AGAP005127 as expected by its integration in the X1 docking site (14). Using nanopore data, genomic deletions in the target gene, AGAP013051, were also confirmed, as expected. The nanopore sequencing data has been deposited to the NCBI sequence read archive (PRJNA862928, reviewer link: dataview.ncbi.nlm.nih.gov/object/PRJNA862928?reviewer-pbmvm5fb9 in78ro2ale7p227oc)

### Transcriptional Profiling and Expression Analysis

To quantify target gene reduction and expression from transgenes as well as to assess global expression patterns, Illumina RNA sequencing was performed. A cross of 50 heavily enriched gFLEG/gFLEG males to 50 homozygous Cas9/Cas9 females was performed to generate gFLEG/Cas9 offspring. Control WT, gFLEG enriched, and Cas9 cages were also prepared. All cages were allowed to mate ad libitum for 5 days and were blood fed. 72 hours after the blood feeding, the egg dish is placed into cages for synchronous egg laying, and females allowed to oviposit for 2 hours. Eggs were collected 18 hours after the first egg lay was observed. Total RNA was extracted using miRNeasy Tissue/Cells Advanced Mini Kit (Qiagen, Cat. No./ID: 217604) from 100 μL embryos, estimate volume, with each genotype (WT; +/Cas9; +/gFLE; gFLE/Cas9) in biological triplicate (12 samples total), following the manufacturer's protocol. Genomic DNA was depleted using the gDNA eliminator column provided by the kit. RNA integrity was assessed using the RNA 6000 Pico Kit for Bioanalyzer (Agilent Technologies, Cat. No./ID: #5067-1513), and mRNA was isolated from ˜1 μg of total RNA using NEBNext Poly (A) mRNA Magnetic Isolation Module (NEB, Cat. No./ID: E7490). RNA-seq libraries were constructed using the NEBNext Ultra II RNA Library Prep Kit for Illumina (NEB, Cat. No./ID: E7770) following the manufacturer's protocols. Briefly, mRNA was fragmented to an average size of 200 nt by incubating at 94° C. for 15 min in the first strand buffer. cDNA was then synthesized using random primers and ProtoScript II Reverse Transcriptase followed by second strand synthesis using NEB Second Strand Synthesis Enzyme Mix. Resulting DNA fragments were end-repaired, dA tailed, and ligated to NEBNext hairpin adaptors (NEB, Cat. No./ID: E7335). Following ligation, adaptors were converted to the “Y” shape by treating with USER enzyme, and DNA fragments were size selected using Agencourt AMPure XP beads (Beckman Coulter #A63880) to generate fragment sizes between 250-350 bp. Adaptor-ligated DNA was PCR amplified followed by AMPure XP bead clean up. Libraries were quantified using a Qubit dsDNA HS Kit (ThermoFisher Scientific, Cat. No./ID: Q32854), and the size distribution was confirmed using a High Sensitivity DNA Kit for Bioanalyzer (Agilent Technologies, Cat. No./ID: 5067-4626). Libraries were sequenced on an Illumina NextSeq2000 in paired end mode with the read length of 50 nt and sequencing depth of 20 million reads per library. Base calls and FASTQ generation were performed with DRAGEN 3.8.4. The reads were mapped to the VectorBase-58_AgambiaePEST genome supplemented with gFLE and Cas9 transgene sequences using STAR. On average, ˜97.4% of the reads were mapped. Gene expression was then quantified using featureCounts against VectorBase annotation release 58 GTF (VectorBase-58_AgambiaePEST.gtf). TPM values were calculated from counts produced by featureCounts and combined. Hierarchical clustering of the data shows that for each genotype, all replicates cluster together, as expected. DESeq2 was then used to perform differential expression analyses between controls (WT; +/Cas9; +/gFLE) and gFLE/Cas9. For each DESeq2 comparison, gene ontology enrichments were performed on significantly differentially expressed genes using R package topGO. Illumina RNA sequencing data has been deposited to the NCBI-SRA, (PRJNA862928, reviewer link: dataview.ncbi.nlm.nih.gov/object/PRJNA862928?reviewer=pbmvm5fb9 in78ro2ale7p227oc)

### Statistical Analysis

Statistical analysis was performed in JMP8.0.2 by SAS Institute Inc and Prism9 for macOS by GraphPad Software, LLC. At least three biological replicates were used to generate statistical means for comparisons. P values were calculated for a two-sided Student's t-test with equal or unequal variance. A two-sided F test was used to assess the variance equality. The departure significance for survival curves was assessed with the Log-rank (Mantel-Cox) and Gehan-Breslow-Wilcoxon texts. Multiple comparisons were corrected by the Bonferroni method. All plots were constructed using Prism 9.1 for macOS by GraphPad Software, LLC.

### Ethical Conduct of Research

All animals were handled in accordance with the Guide for the Care and Use of Laboratory Animals as recommended by the National Institutes of Health and approved by the UCSD Institutional Animal Care and Use Committee (IACUC, Animal Use Protocol #S17187) and UCSD Biological Use Authorization (BUA #R2401).

### Data Availability

Complete sequence maps and plasmids are deposited at Addgene.org (#187238). All Illumina and Nanopore sequencing data has been deposited to the NCBI-SRA PRJNA862928, reviewer link: dataview.ncbi.nlm.nih.gov/object/PRJNA862928?reviewer=pbmvm5fb9 in78ro2ale7p227oc. Generated A. gambiae transgenic lines are available upon request to O.S.A.

It will be understood from the foregoing description that various modifications and changes may be made in the various embodiments of the present disclosure without departing from their true spirit. The description provided herein is intended for purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be a useful and readily understood description of procedures as well as of the principles and conceptual aspects of the inventive concepts. Changes may be made in the construction and formulation of the various components and compositions described herein, the methods described herein or in the steps or the sequence of steps of the methods described herein without departing from the spirit and scope of the presently disclosed inventive concepts.

### REFERENCES

1. Organization, W. H. & Others. World malaria report 2020:20 years of global progress and challenges. (2020).
2. Ranson, H. & Lissenden, N. Insecticide Resistance in African Anopheles Mosquitoes: A Worsening Situation that Needs Urgent Action to Maintain Malaria Control. Trends Parasitol. 32, 187-196 (2016).
3. Haddow, A. J. & Ssenkubuge, Y. The mosquitoes of Bwamba County, Uganda. IX. Further studies on the biting behaviour of an outdoor population of the Anopheles gambiae Giles complex. Bull. Entomol. Res. 62, 407-414 (1973).
4. Quiñones, M. L. et al. Anopheles gambiae gonotrophic cycle duration, biting and exiting behaviour unaffected by permethrin-impregnated bednets in The Gambia. Med. Vet. Entomol. 11, 71-78 (1997).
5. Sougoufara, S., Ottih, E. C. & Tripet, F. The need for new vector control approaches targeting outdoor biting Anopheline malaria vector communities. Parasit. Vectors 13, 295 (2020).
6. Papathanos, P. A. et al. Sex separation strategies: past experience and new approaches. Malar. J. 8 Suppl 2, S5 (2009).
7. Gilles, J. R. L. et al. Towards mosquito sterile insect technique programmes: exploring genetic, molecular, mechanical and behavioural methods of sex separation in mosquitoes. Acta Trop. 132 Suppl, S178-87 (2014).
8. Bourtzis, K. & Tu, Z. J. Joint FAO/IAEA Coordinated Research Project on ‘Exploring genetic, molecular, mechanical and behavioural methods of sex separation in mosquitoes’—an introduction. Parasit. Vectors 11, 653 (2018).
9. Gunathilaka, N. et al. Use of mechanical and behavioural methods to eliminate female Aedes aegypti and Aedes albopictus for sterile insect technique and incompatible insect technique applications. Parasit. Vectors 12, 148 (2019).
10. Zacarés, M. et al. Exploring the potential of computer vision analysis of pupae size dimorphism for adaptive sex sorting systems of various vector mosquito species. Parasit. Vectors 11, 656 (2018).
11. Yamada, H. et al. The Anopheles arabiensis genetic sexing strain ANO IPCLI and its application potential for the sterile insect technique in integrated vector management programmes. Acta Trop. 142, 138-144 (2015).
12. Taracena, M. L., Hunt, C. M., Benedict, M. Q., Pennington, P. M. & Dotson, E. M. Downregulation of female doublesex expression by oral-mediated RNA interference reduces number and fitness of Anopheles gambiae adult females. Parasit. Vectors 12, 170 (2019).
13. Bernardini, F. et al. Site-specific genetic engineering of the Anopheles gambiae Y chromosome. Proc. Natl. Acad. Sci. U.S.A 111, 7600-7605 (2014).
14. Volohonsky, G. et al. Tools for Anopheles gambiae Transgenesis. G3 5, 1151-1163 (2015).
15. Lutrat, C. et al. Combining two Genetic Sexing Strains allows sorting of non-transgenic males for Aedes genetic control. bioRxiv 2022.03.11.483912 (2022) doi: 10.1101/2022.03.11.483912.
16. Catteruccia, F., Benton, J. P. & Crisanti, A. An Anopheles transgenic sexing strain for vector control. Nat. Biotechnol. 23, 1414-1417 (2005).
17. Smidler, A. L., Scott, S. N., Mameli, E., Shaw, W. R. & Catteruccia, F. A transgenic tool to assess Anopheles mating competitiveness in the field. Parasit. Vectors 11, 651 (2018).
18. Marois, E. et al. High-throughput sorting of mosquito larvae for laboratory studies and for future vector control interventions. Malaria Journal vol. 11 (2012).
19. Magnusson, K. et al. Transcription regulation of sex-biased genes during ontogeny in the malaria vector Anopheles gambiae. PLOS One 6, e21572 (2011).
20. Kandul, N. P. et al. Transforming insect population control with precision guided sterile males with demonstration in flies. Nat. Commun. 10, 84 (2019).
21. Dafa′alla, T. et al. A self-limiting, sex-specific gene and methods of using. World Patent (2018).
22. Li, M. et al. Eliminating Mosquitoes with Precision Guided Sterile Males. 33 (2021).
23. Galizi, R. et al. A synthetic sex ratio distortion system for the control of the human malaria mosquito. Nat. Commun. 5, 3977 (2014).
24. Galizi, R. et al. A CRISPR-Cas9 sex-ratio distortion system for genetic control. Sci. Rep. 6, 31139 (2016).
25. Windbichler, N., Papathanos, P. A. & Crisanti, A. Targeting the X chromosome during spermatogenesis induces Y chromosome transmission ratio distortion and early dominant embryo lethality in Anopheles gambiae. PLOS Genet. 4, e1000291 (2008).
26. Klein, T. A., Windbichler, N., Deredec, A., Burt, A. & Benedict, M. Q. Infertility resulting from transgenic I-PpoI male Anopheles gambiae in large cage trials. Pathog. Glob. Health 106, 20-31 (2012).
27. Yao, F. A. et al. Mark-release-recapture experiment in Burkina Faso demonstrates reduced fitness and dispersal of genetically-modified sterile malaria mosquitoes. Nat. Commun. 13, 796 (2022).
28. Krzywinska, E. & Krzywinski, J. Effects of stable ectopic expression of the primary sex determination gene Yob in the mosquito Anopheles gambiae. Parasit. Vectors 11, 648 (2018).
29. Papathanos, P. A. et al. A perspective on the need and current status of efficient sex separation methods for mosquito genetic control. Parasit. Vectors 11, 654 (2018).
30. Kyrou, K. et al. A CRISPR-Cas9 gene drive targeting doublesex causes complete population suppression in caged Anopheles gambiae mosquitoes. Nat. Biotechnol. 36, 1062-1066 (2018).
31. Simoni, A. et al. A male-biased sex-distorter gene drive for the human malaria vector Anopheles gambiae. Nat. Biotechnol. 38, 1054-1060 (2020).
32. Beaghton, A. K., Hammond, A., Nolan, T., Crisanti, A. & Burt, A. Gene drive for population genetic control: non-functional resistance and parental effects. Proc. Biol. Sci. 286, 20191586 (2019).
33. Krzywinska, E. et al. femaleless Controls Sex Determination and Dosage Compensation Pathways in Females of Anopheles Mosquitoes. Current Biology vol. 31 1084-1091.e4 (2021).
34. Werling, K. et al. Steroid Hormone Function Controls Non-competitive Plasmodium Development in Anopheles. Cell 177, 315-325.e14 (2019).
35. Valsecchi, C. I. K., Marois, E., Basilicata, M. F., Georgiev, P. & Akhtar, A. Distinct mechanisms mediate X chromosome dosage compensation in Anopheles and Drosophila. Life Science Alliance 4, (2021).
36. A. L. Smidler, O. Terenzi, J. Soichot, E. A. Levashina, E. Marois, Targeted mutagenesis in the malaria mosquito using TALE nucleases. PLOS One. 8, e74511 (2013).
37. M. Li, T. Yang, M. Bui, S. Gamez, T. Wise, N. P. Kandul, J. Liu, L. Alcantara, H. Lee, J. R. Edula, R. Raban, Y. Zhan, Y. Wang, N. DeBeaubien, J. Chen, H. M. Sánchez C, J. B. Bennett, I. Antoshechkin, C. Montell, J. M. Marshall, O. S. Akbari, Suppressing mosquito populations with precision guided sterile males. Nat. Commun. 12, 5374 (2021).
38. H. M. S. C., H. M. Sánchez C., S. L. Wu, J. B. Bennett, J. M. Marshall, MGD riv E: A modular simulation framework for the spread of gene drives through spatially explicit mosquito populations. Methods in Ecology and Evolution. 11 (2020), pp. 229-239.
39. D. O. Carvalho, A. R. McKemey, L. Garziera, R. Lacroix, C. A. Donnelly, L. Alphey, A. Malavasi, M. L. Capurro, Suppression of a Field Population of Aedes aegypti in Brazil by Sustained Release of Transgenic Male Mosquitoes. PLOS Negl. Trop. Dis. 9, e0003864 (2015).
40. J.-M. O. Depinay, C. M. Mbogo, G. Killeen, B. Knols, J. Beier, J. Carlson, J. Dushoff, P. Billingsley, H. Mwambi, J. Githure, A. M. Toure, F. E. Mckenzie, A simulation model of African Anopheles ecology and population dynamics for the analysis of malaria transmission. Malar. J. 3, 29 (2004).
41. G. M. Chambers, M. J. Klowden, Age of Anopheles gambiae Giles male mosquitoes at time of mating influences female oviposition. J. Vector Ecol. 26, 196-201 (2001).
42. Sawadogo, Diabaté, Toé, Effects of Age and Size on Anopheles gambiae ss Male Mosquito Mating Success. J. Med. Surg. Pathol. (available at academic.oup.com/jme/article-abstract/50/2/285/853180).
43. E. Krzywinska, N. J. Dennison, G. J. Lycett, J. Krzywinski, A maleness gene in the malaria mosquito Anopheles gambiae. Science. 353, 67-69 (2016).
44. R. Raban, W. A. C. Gendron, O. S. Akbari, A perspective on the expansion of the genetic technologies to support the control of neglected vector-borne diseases and conservation. Frontiers in Tropical Diseases. 3 (2022), doi: 10.3389/fitd.2022.999273.
45. E. F. Knipling, Sterile-Male Method of Population Control. Science. 130 (1959), pp. 902-904.
46. G. Munhenga, B. D. Brooke, J. R. L. Gilles, K. Slabbert, A. Kemp, L. C. Dandalo, O. R. Wood, L. N. Lobb, D. Govender, M. Renke, L. L. Koekemoer, Mating competitiveness of sterile genetic sexing strain males (GAMA) under laboratory and semi-field conditions: Steps towards the use of the Sterile Insect Technique to control the major malaria vector Anopheles arabiensis in South Africa. Parasites & Vectors. 9 (2016), doi: 10.1186/s13071-016-1385-9.
47. A. Hammond, R. Galizi, K. Kyrou, A. Simoni, C. Siniscalchi, D. Katsanos, M. Gribble, D. Baker, E. Marois, S. Russell, A. Burt, N. Windbichler, A. Crisanti, T. Nolan, A CRISPR-Cas9 gene drive system targeting female reproduction in the malaria mosquito vector Anopheles gambiae. Nat. Biotechnol. 34, 78-83 (2016).
48. S. Halliday, Oxitec successfully completes first field deployment of 2nd Generation friendlyIM Aedes aegypti technology. Oxitec (2019), (available at www.oxitec.com/en/news/oxitec-successfully-completes-first-field-deployment-of-2nd-generation-friendly-aedes-aegypti-technology).
49. J. Romeis, J. Collatz, D. C. M. Glandorf, M. B. Bonsall, The value of existing regulatory frameworks for the environmental risk assessment of agricultural pest control using gene drives. Environ. Sci. Policy. 108, 19-36 (2020).
50. M. Q. Benedict, The MR4 Methods in Anopheles research laboratory manual. Atlanta: CDC; 2007 (2018).
51. M. Scott, Sex Determination and Dosage Compensation: femaleless Is the Link in Anopheles Mosquitoes. Curr. Biol. 31 (2021), pp. R260-R263.
52. K. M. Esvelt, A. L. Smidler, F. Catteruccia, G. M. Church, Concerning RNA-guided gene drives for the alteration of wild populations. Elife. 3 (2014), doi: 10.7554/eLife.03401.
53. J. Champer, A. Buchman, O. S. Akbari, Cheating evolution: engineering gene drives to manipulate the fate of wild populations. Nat. Rev. Genet. 17, 146-159 (2016).
54. N. P. Kandul, J. Liu, J. B. Bennett, J. M. Marshall, O. S. Akbari, A confinable home-and-rescue gene drive for population modification. Elife. 10 (2021), doi: 10.7554/eLife.65939.
55. J. Hendrichs, G. Franz, P. Rendon, Increased effectiveness and applicability of the sterile insect technique through male-only releases for control of Mediterranean fruit flies during fruiting seasons. J. Appl. Entomol. 119, 371-377 (1995).
56. A. Smidler, CRISPR-based innovative genetic tools for control of Anopheles gambiae mosquitoes. Thesis, Harvard University (2019) (available at dash.harvard.edu/handle/1/42029729).
57. J. Krzywinski, D. R. Nusskern, M. K. Kern, N. J. Besansky, Isolation and characterization of Y chromosome sequences from the African malaria mosquito Anopheles gambiae. Genetics. 166, 1291-1302 (2004).
58. R. J. Kent, A. J. West, D. E. Norris, Molecular differentiation of colonized human malaria vectors by 28S ribosomal DNA polymorphisms. Am. J. Trop. Med. Hyg. 71, 514-517 (2004).
59. A. Deredec, H. C. J. Godfray, A. Burt, Requirements for effective malaria control with homing endonuclease genes. Proc. Natl. Acad. Sci. U.S.A 108, E874-80 (2011).
60. World Health Organization (WHO), Garki project: Research on the epidemiology and control of malaria in the Sudan Savanna of west Africa (World Health Organization, Genève, Switzerland, 1980).
61. C. Taylor, Y. T. Touré, J. Carnahan, D. E. Norris, G. Dolo, S. F. Traoré, F. E. Edillo, G. C. Lanzaro, Gene flow among populations of the malaria vector, Anopheles gambiae, in Mali, West Africa. Genetics. 157, 743-750 (2001).

What is claimed is:

1. A transgenic animal, comprising:
a first transgene encoding a Cas9 protein; and
a second transgene encoding a guide RNA (gRNA) that targets a female essential gene,
wherein the first transgene and the second transgene are incorporated into a genome of the transgenic animal, and wherein the second transgene is incorporated into the genome at a location which does not encode the female essential gene.

2. The transgenic animal of claim 1, wherein the transgenic animal is a male.

3. The transgenic animal of claim 1, wherein the animal is an insect.

4. The transgenic animal of claim 3, wherein the insect is a mosquito.

5. The transgenic animal of claim 4, wherein the mosquito is A. gambiae.

6. The transgenic animal of claim 4, wherein the female essential gene is implicated in regulation of the dsx gene.

7. The transgenic animal of claim 4, wherein the female essential gene is fle.

8. The transgenic animal of claim 1, wherein the transgenic animal is reproductively viable.

9. The transgenic animal of claim 1, wherein the presence of the first transgene and the second transgene in a female animal is lethal to the animal.

10. The transgenic animal of claim 9, wherein the presence of the first transgene and the second transgene in the female animal is lethal at a larval or pupal stage of development.

11. The transgenic animal of claim 1, wherein the first transgene and the second transgene are incorporated at different positions of the genome.

12. The transgenic animal of claim 1, wherein the Cas9 protein is a Vasa2-Cas9 protein.

13. The transgenic animal of claim 1, wherein mating of the transgenic animal with a wild type animal produces offspring which contain the first transgene, the second transgene, or both.

14. The transgenic animal of claim 1, wherein mating of the transgenic animal with a wild type animal produces offspring which contain a somatic and/or heritable germline mutation of the female essential gene.

15. The transgenic animal of claim 1, wherein mating of the transgenic animal with a wild type animal produces offspring which include female essential gene mutant males reproductively viable to pass along the female essential gene mutation into subsequent generations.

16. The transgenic animal of claim 1, wherein the transgenic animal is a male mosquito and the female essential gene is fle.

17. A method of suppressing a population of animals, comprising:
releasing a first population of the transgenic animals of claim 1 into the population of animals; and
allowing the transgenic animals to mate with the population of animals, thereby passing the first transgene and the second transgene into subsequent generations.

18. The method of claim 17, further comprising releasing additional populations of the transgenic animals and allowing the transgenic animals to mate with the population of animals and the subsequent generations, thereby suppressing the population of animals due to the production of non-viable female offspring.

19. A method of preparing a transgenic animal for suppressing a population of animals, comprising:
providing a first parental transgenic animal strain comprising a transgene encoding a Cas9 protein;
providing a second parental transgenic animal strain comprising a second transgene encoding a guide RNA (gRNA) that targets a female essential gene, wherein the second transgene is incorporated into a genome at a position which does not encode the female essential gene; and
sexually crossing the first and second parental transgenic animal strains to produce offspring.

20. The method of claim 19, wherein the offspring include animals which encode the Cas9 protein, the gRNA, or both.

21. The method of claim 19, wherein the offspring include animals which comprise a somatic and/or heritable germline mutation in the female essential gene.

22. The method of claim 19, wherein mating of the offspring with a wild type animal passes along a female essential gene mutation in reproductively viable males.

23. The method of claim 19, wherein the first parental transgenic animal strain is homozygous for the transgene encoding of the Cas9 protein, the second parental transgenic animal strain is homozygous for the transgene encoding of the gRNA, or both are homozygous for their respective transgenes.

24. The method of claim 19, wherein all or substantially all of the offspring which are viable are males which comprise the transgene encoding the gRNA, the transgene encoding the Cas9 protein, and/or heritable germline mutations in the female essential gene.

25. A transgenic system for population control of an animal comprising:
a first parental transgenic animal strain comprising a first transgene encoding a Cas9 protein;
a second parental transgenic animal strain comprising a second transgene encoding a guide RNA (gRNA) that targets a female essential gene, wherein the second transgene is incorporated into a genome at a location which does not encode the female essential gene;
wherein genetic crossing of the two strains results in offspring which:
include animals which encode the Cas9 protein, the gRNA, or both;
include animals which comprise a somatic and/or heritable germline mutation in the female essential gene; and/or
include viable males capable of passing along a female essential gene mutation to subsequent offspring.

26. The transgenic system of claim 25, wherein the first parental transgenic animal strain is homozygous for the Cas9 protein and wherein the second parental transgenic animal strain is homozygous for the gRNA.
